(19) United States
(12) Patent Application Publication
(10) Pub. No.: US 2020/0185604 A1
(43) Pub. Date: Jun. 11, 2020

(54) COMPOSITION CONTAINING AMINIUM RADICAL CATION

(71) Applicant: Dow Global Technologies LLC, Midland, MI (US)

(72) Inventors: Robert David Grigg, Midland, MI (US); Liam P. Spencer, Lake Jackson, TX (US); John W. Kramer, Midland, MI (US); David D. Devore, Midland, MI (US); Brian Goodfellow, Midland, MI (US); Chun Liu, Midland, MI (US); Sukrit Mukhopadhyay, Midland, MI (US); Thomas H. Peterson, Midland, MI (US); William H. H. Woodward, Harbor Beach, MI (US); Anatoliy N. Sokolov, Midland, MI (US)

(21) Appl. No.: 16/329,836

(22) PCT Filed: Oct. 20, 2017
(86) PCT No.: PCT/US2017/057495

§ 371 Date: Mar. 1, 2019

Related U.S. Application Data
(60) Provisional application No. 62/417,533, filed on Nov. 4, 2016.

(51) Int. Cl. H01L 51/00; C07D 209/86; C09K 11/06; C07C 211/61

(52) U.S. Cl. CPC H01L 51/006; C07D 209/86; H01L 51/5056; H01L 51/0061; C07C 211/61; C09K 11/06

(57) ABSTRACT

Provided is an organic light-emitting diode comprising a substrate, an anode layer, optionally one or more hole injection layers, one or more hole transport layers, optionally one or more electron blocking layers, an emitting layer, optionally one or more hole blocking layers, optionally one or more electron transport layers, an electron injection layer, and a cathode, wherein either the hole injection layer, or the hole transport layer, or both of the hole injection layer and the hole transport layer, or a layer that functions as both a hole injection layer and a hole transport layer, comprises a polymer that comprises one or more triaryl aminium radical cations having the structure (S1) wherein each of R¹¹, R¹², R¹³, R¹⁴, R¹⁵, R²¹, R²², R²³, R²⁴, R²⁵, R³¹, R³², R³³, R³⁴, and R³⁵ is independently selected from the group consisting of hydrogen, deuterium halogens, amine groups, hydroxyl groups, sulfonate groups, nitro groups, and organic groups, wherein two or more of R¹¹, R¹², R¹³, R¹⁴, R¹⁵, R²¹, R²², R²³, R²⁴, R²⁵, R³¹; R³²; R³³ R³⁴ and R³⁵ are optionally connected to each other to form a ring structure; wherein one or more of R¹¹, R¹², R¹³, R¹⁴, R¹⁵, R²¹, R²², R²³, R²⁴, R²⁵, R³¹, R³², R³³, R³⁴, and R³⁵ is covalently bound to the polymer, and wherein A″ is an anion.

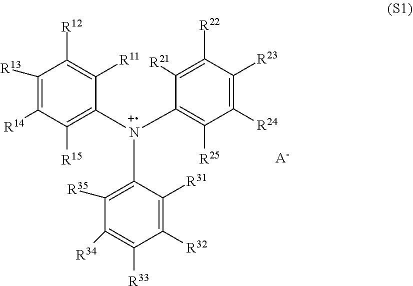

Many opto-electronic devices are multilayer compositions. For example, organic light-emitting diodes (OLEDs) normally contain multiple layers, including, among other layers, an emitting layer and either or both of a hole transport layer (HTL) or a hole injection layer (HIL). A desirable method of creating an HTL or an HIL is to apply a layer of a solution of the HTL or HIL material in a solvent and then evaporate the solvent. Among compositions suitable for use in an HTL or an HIL that will be applied by such a solution method, it is desirable that the composition has one or more of the following characteristics. The composition should readily transport holes; the composition should dissolve readily in one or more organic solvents; the composition should be capable of being deposited in a layer by first depositing a layer of a solution containing the composition and a solvent and then evaporating the solvent; a layer of the composition, when dried, should be resistant to removal by one or more hydrocarbon solvents; no portion of the composition should readily migrate to other layers of the opto-electronic device. When an OLED is made using such a composition, it is desirable that the OLED have high efficiency and/or operate at a low drive voltage.

A. Yamamori et al., in Applied Physics Letters, vol. 72, pp. 2147-2149 (1998) describe a hole transport layer containing a matrix polycarbonate polymer and a dopant molecule, tris(4-bromoethyl)aminium hexachloroantimonate (TBAHA), which is not covalently bound to the matrix polymer. It is considered that, in such layers where the dopant is not bound, the dopant is susceptible to migration to other layers, such as the emitting layer, of an opto-electronic device.

The following is a statement of the invention.

A first aspect of the present invention is a composition comprising a polymer that comprises one or more triaryl aminium radical cations having the structure (S1)

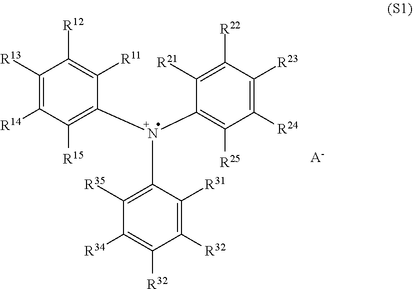

wherein each of R¹¹, R¹², R¹³, R¹⁴, R¹⁵, R²¹, R²², R²³, R²⁴, R²⁵, R³¹, R³², R³³, R³⁴, and R³⁵ is independently H or deuterium or an organic group, wherein two or more of R¹¹, R¹², R¹³, R¹⁴, R¹⁵, R²¹, R²², R²³, R²⁴, R²⁵, R³¹, R³², R³³, R³⁴, and R³⁵ are optionally connected to each other to form a ring structure; and A⁻ is an anion,
wherein one or more of R¹¹, R¹², R¹³, R¹⁴, R¹⁵, R²¹, R²², R²³, R²⁴, R²⁵, R³¹, R³², R³³, R³⁴, and R³⁵ is covalently bound to the polymer.

A second aspect of the present invention is an organic light-emitting diode comprising, an anode layer, optionally one or more hole injection layers, one or more hole transport layers, optionally one or more electron blocking layers, an emitting layer, optionally one or more hole blocking layers, optionally one or more electron transport layers, an electron injection layer, and a cathode, wherein either a hole injection layer, or a hole transport layer, or both of a hole injection layer and a hole transport layer, or a layer that functions as both a hole injection layer and a hole transport layer, comprises a polymer as described in the first aspect.

The following is a brief description of the drawing.

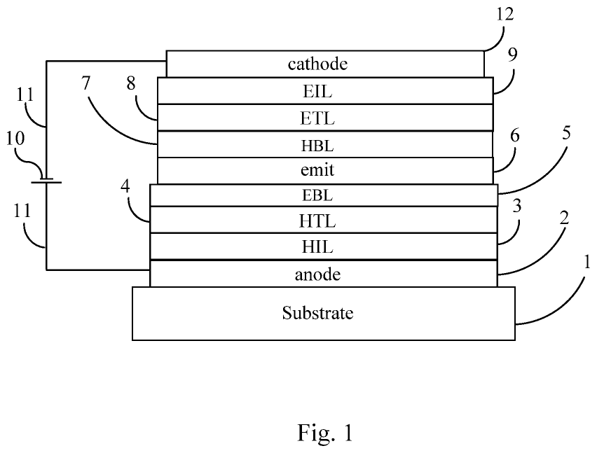

FIG. 1 shows one embodiment of an OLED made using a composition of the present invention.

The following is a detailed description of the invention.

As used herein, the following terms have the designated definitions, unless the context clearly indicates otherwise.

The term “alkoxy group,” as described herein, refers to an alkyl group in which at least one hydrogen atom is substituted with an oxygen atom, O.

The term “alkyl group,” as described herein, refers to an organic radical derived from an alkyl hydrocarbon molecule by deleting one hydrogen atom therefrom. When a chemical group is referred to herein as “an alkyl,” it is meant that that chemical group is an alkyl group. An alkyl group may be linear, branched, cyclic or a combination thereof. The term “substituted alkyl,” as used herein, refers to an alkyl, in which at least one hydrogen atom is substituted with a substituent that comprises at least one heteroatom. Heteroatoms include, but are not limited to, O, N, P and S. Substituents include, but are not limited to, halide, OR′, NR′₂, PR′₂, P(═O)R′₂, SiR′₃; where each R′ is independently a C₁-C₂₀ hydrocarbyl group.

The “anode” injects holes into either the emitting layer or a layer that is located between the emitting layer and the anode, such as a hole injection layer or a hole transport layer. The anode is disposed on a substrate. The anode is typically made from a metal, a metal oxide, a metal halide, an electroconductive polymer, or combinations thereof.

The term “aryl group,” as described herein, refers to an organic radical derived from aromatic hydrocarbon molecule by deleting one hydrogen atom therefrom. An aryl group may be a monocyclic and/or fused ring system, each ring of which suitably contains from 5 to 7, preferably from 5 to 6 atoms. Structures wherein two or more aryl groups are combined through single bond(s) are also included. Specific examples include, but are not limited to, phenyl, tolyl, naphthyl, biphenyl, anthryl, indenyl, fluorenyl, benzofluorenyl, phenanthryl, triphenylenyl, pyrenyl, perylenyl, chrysenyl, naphtacenyl, fluoranthenyl, and the like. The naphthyl may be 1-naphthyl or 2-naphthyl, the anthryl may be 1-anthryl, 2-anthryl or 9-anthryl, and the fluorenyl may be any one of 1-fluorenyl, 2-fluorenyl, 3-fluorenyl, 4-fluorenyl and 9-fluorenyl. The term “substituted aryl,” as used herein, refers to an aryl, in which at least one hydrogen atom is substituted with a substituent comprising at least one heteroatom, or a substituent comprising at least one substituted or unsubstituted alkyl group, or any combination thereof. Heteroatoms include, but are not limited to, O, N, P and S. Substituents include, but are not limited to, halide, OR′, NR′₂, PR′₂, P(═O)R′₂, SiR′₃; where each R′ is independently a C₁-C₂₀ hydrocarbyl group. This definition of “substituted aryl” applies to any group that contains an aromatic ring, such as, for example, phenyl, carbazolyl, indolyl, fluorenyl, and biphenyl.

The term “aryloxy,” as described herein, refers to an aryl in which at least one hydrogen atom is replaced with an oxygen atom, O.

The term “amine” as described herein refers to a compound having one or more amine nitrogen atoms. An amine nitrogen atom is a nitrogen atom that is part of a structure R⁴¹NH₂, R⁴¹R⁴²NH, or R⁴¹R⁴²R⁴³N, where each of R⁴¹, R⁴², and R⁴³ is a substituted or unsubstituted alkyl or aryl group. R⁴¹, R⁴², and R⁴³ may be separate groups, or any two or more of R⁴¹, R⁴², and R⁴³ may be connected to each other to form one or more aromatic rings or one or more aliphatic rings or a combination thereof. An amine may have exactly one amine nitrogen atom or may have two or more amine nitrogen atoms. An amine having one or more aromatic rings is an aromatic amine.

As used herein, and as would be understood by one skilled in the art, the term “blocking layer” means that the layer provides a barrier that significantly inhibits transport of one type of charge carriers and/or excitons through the device, without suggesting that the layer necessarily completely blocks all charge carriers and/or excitons. The presence of such a blocking layer in a device may result in higher efficiencies as compared to a similar device lacking a blocking layer. Also, a blocking layer may be used to confine emission to a desired region of an OLED. Blocking layers, when present, are generally present on either side of the emitting layer.

Electron blocking may be accomplished in various ways including, for example, by using a blocking layer that has a LUMO energy level that is significantly higher than the LUMO energy level of the emissive layer. The greater difference in LUMO energy levels results in better electron blocking properties. Suitable materials for use in the blocking layer are dependent upon the material of emissive layer. A layer that primarily performs electron blocking is an electron blocking layer (EBL). Electron blocking may occur in other layers, for example, a hole transport layer (HTL).

Hole blocking may be accomplished in various ways including, for example, by using a blocking layer that has a HOMO energy level that is significantly lower than the HOMO energy level of the emissive layer. The greater difference in HOMO energy levels results in better hole blocking properties. Suitable materials for use in the blocking layer are dependent upon the material of emissive layer. A layer that primarily performs hole blocking is a hole blocking layer (HBL). Hole blocking may occur in other layers, for example, an electron transport layer (ETL).

Blocking layers may also be used to block excitons from diffusing out of the emissive layer by using a blocking layer that has a triplet energy level that is significantly higher than the triplet energy level of the EML dopant or the EML host. Suitable materials for use in the blocking layer are dependent upon the material composition of emissive layer.

The “cathode” injects electrons into either the emitting layer or a layer that is located between the emitting layer and the cathode, such as an electron injection layer or an electron transport layer. The cathode is typically made from a metal, a metal oxide, a metal halide, an electroconductive polymer, or a combination thereof.

“Dopant” and like terms, refer to a material that is present in a layer in relatively small amounts, normally 10% or less by weight based on the weight of the layer. The dopant is usually distributed statistically throughout the layer. The dopant is present to provide desired electrical properties to the layer. The term “dopant” herein refers to a molecule that is not a polymer.

“Electron injection layer,” or “EIL,” and like terms is a layer which improves injection of electrons injected from the cathode into the electron transport layer.

“Electron transport layer (or “ETL”),” and like terms, refers to a layer made from a material, which exhibits properties including high electron mobility for efficiently transporting electrons injected from the cathode or the EIL and favorable injection of those electrons into the hole blocking layer or the emitting layer.

“Electron Volt” or “eV” is the amount of energy gained (or lost) by the charge of a single electron moved across an electric potential difference of one volt.

“Emitting layer” and like terms, is a layer located between electrodes (anode and cathode) and is the primary light-emitting source. The emitting layer typically consists of host and emitter. The host material could be preferentially hole or electron transporting or can be similarly transporting of both holes and electrons, and may be used alone or by combination of two or more host materials. The opto-electrical properties of the host material may differ to which type of emitter (Phosphorescent or Fluorescent) is used. The emitter is a material that undergoes radiative emission from an excited state. The excited state can be generated, for example, by charges on the emitter molecule or by energy transfer from the excited state of another molecule.

The term “heteroalkyl,” as described herein, refers to an alkyl group, in which at least one carbon atom or CH group or CH₂ is substituted with a heteroatom or a chemical group containing at least one heteroatom. Heteroatoms include, but are not limited to, O, N, P and S. A heteroalkyl group may be linear, branched, cyclic or a combination thereof. The term “substituted heteroalkyl,” as used herein, refers to a heteroalkyl, in which at least one hydrogen atom is substituted with a substituent that comprises at least one heteroatom. Heteroatoms include, but are not limited to, O, N, P and S. Substituents include, but are not limited to, halide, OR″, NR″₂, PR″₂, P(═O)R″₂, SiR″₃; where each R″ is independently a C₁-C₂₀ hydrocarbyl group.

The term “heteroaryl,” as described herein, refers to an aryl group, in which at least one carbon atom or CH group or CH₂ of an aromatic ring is replaced with a heteroatom or a chemical group containing at least one heteroatom. Heteroatoms include, but are not limited to, O, N, P, and S. The heteroaryl may be a 5- or 6-membered monocyclic heteroaryl or a polycyclic heteroaryl which is fused with one or more benzene ring(s), and may be partially saturated. The structures having one or more heteroaryl group(s) bonded through a single bond are also included. The heteroaryl groups may include divalent aryl groups of which the heteroatoms are oxidized or quarternized to form N-oxides, quaternary salts, or the like. The term “substituted heteroaryl,” as used herein, refers to a heteroaryl in which at least one hydrogen atom is substituted with a substituent composed of an unsubstituted alkyl, a substituted alkyl, at least one heteroatom, and any combination thereof. Heteroatoms include, but are not limited to, O, N, P, and S. Substituents include, but are not limited to, halide, OR′, NR′₂, PR′₂, P(═O)R′₂, SiR′₃; where each R′ is independently a C₁-C₂₀ hydrocarbyl group.

A “heteroatom” is an atom other than carbon or hydrogen. Nonlimiting examples of heteroatoms include: F, Cl, Br, N, O, P, B, S, Si, Sb, Al, Sn, As, Se, and Ge.

“Hole injection layer,” or “HIL,” and like terms, is a layer for efficiently transporting or injecting holes from the anode into the emissive layer, the electron blocking layer, or more typically into the hole transport layer. Multiple hole injection layers may be used to accomplish hole injection from the anode to the hole transporting layer, electron blocking layer or the emitting layer.

“Hole transport layer (or “HTL”),” and like terms, refers to a layer made from a material, which exhibits properties including high hole mobility for efficiently transporting holes injected from the anode or the HIL and favorable injection of those holes into the electron blocking layer or the emitting layer.

The term “hydrocarbon,” as used herein, refers to a chemical group containing only hydrogen atoms and carbon atoms. The term “hydrocarbon” includes “a hydrocarbyl” which is a hydrocarbon substituent having a valence (typically univalent). The term “substituted hydrocarbon,” (or “substituted hydrocarbyl”), as used herein, refers to a hydrocarbon (or hydrocarbyl) in which at least one hydrogen atom is substituted with a substituent comprising at least one heteroatom. An “unsubstituted hydrocarbon” (or “unsubstituted hydrocarbyl”) is a hydrocarbon that contains no heteroatoms.

The term “organic group” refers to a chemical group that contains one or more carbon atoms and also contains one or more atoms of an element other than carbon, which may be, for example, hydrogen, halogen, nitrogen, oxygen, sulfur, phosphorous, or another element, or a combination thereof.

The term “phenyl group” means a group that has structure (S3):

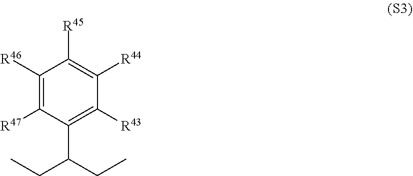

A phenyl group has a single point of attachment to another molecule. The point of attachment is denoted in groups of chemical structures herein by the jagged line symbol [symbol] . In an “unsubstituted phenyl group,” each of R⁴³ through R⁴⁷ is hydrogen. In a “substituted phenyl group,” one or more of R⁴³ through R⁴⁷ is an atom or group other than hydrogen. Each of R⁴³ through R⁴⁷ is independently hydrogen or a substituted or unsubstituted hydrocarbyl group. Any two or more of R⁴³ through R⁴⁷ may be connected to each other to form a ring structure, which may be aliphatic, aromatic, or a combination thereof, and which may contain a single ring or multiple rings. Each of R⁴³ through R⁴⁷ optionally contains one or more heteroatoms other than carbon and hydrogen.

A “ring structure,” as used herein, is a chemical group that contains three or more atoms covalently bonded to each other in such a way that at least one path can be traced along covalent bonds from a first atom, through two or more other atoms, and back to the first atom. A ring structure may contain carbon, hydrogen, one or more atoms other than carbon and hydrogen, or a combination thereof. A ring structure can be saturated or unsaturated, including aromatic, and the ring structure can contain one, or two, or more than two rings.

The “substrate” is a support for the organic light-emitting device. Nonlimiting examples of material suitable for the substrate include quartz plate, glass plate, metal plate, metal foil, plastic film from polymeric resins such as polyester, polymethacrylate, polycarbonate, and polysulfone.

A “polymer,” as used herein is a relatively large molecule made up of the reaction products of smaller chemical repeat units. Polymers may have structures that are linear, branched, star shaped, looped, hyperbranched, crosslinked, or a combination thereof; polymers may have a single type of repeat unit (“homopolymers”) or they may have more than one type of repeat unit (“copolymers”). Copolymers may have the various types of repeat units arranged randomly, in sequence, in blocks, in other arrangements, or in any mixture or combination thereof.

Polymer molecular weights can be measured by gel permeation chromatography (GPC). Polymers have number-average molecular weight of 2500 Da or more.

Molecules that can react with each other to form the repeat units of a polymer are known herein as “monomers.” The repeat units so formed are known herein as “polymerized units” of the monomer.

Various types of polymers are defined by the chemical reaction that bonds the monomers together. Vinyl polymers result from vinyl group on one monomer reacting with a vinyl group on another monomer. A vinyl group contains a non-aromatic carbon-carbon double bond. Polyurethanes result from an isocyanate group on one monomer reacting with an isocyanate-reactive group on another monomer; isocyanate-reactive groups include hydroxyl groups (including the OH group in water), amine groups, and carboxyl groups. Polyamides result from a carboxyl group on one monomer reacting with an amine group on another monomer. Epoxy polymers result from an epoxy group on one monomer reacting with a hydroxyl group on another monomer. Polyesters result from a carboxyl group on one monomer reacting with a hydroxyl group on another monomer.

Another type of polymer are the conjugated polymers. Conjugated polymers have repeat units that are conjugated structures. Conjugated structures include structures with aromatic rings connected to each other with a carbon-carbon single bond, structures with an aromatic ring connected by a single bond to a nitrogen atom that is in turn connected to another aromatic ring by a single bond, linear structures with alternating carbon-carbon double bonds and carbon-carbon single bonds, and combinations thereof. A conjugated structure in the repeat unit may or may not have one or more substituent groups pendant from it. Repeat units are considered to be joined by sp² hybridized carbon-carbon single bonds.

A “complementary” pair of reactive groups is a pair of reactive groups (G1 and G2) that can react with each other in a polymerization reaction. Some exemplary complementary pairs of reactive groups are as follows:

| G1 | G2 | Polymer Type |
|---|---|---|
| isocyanate | hydroxyl, amine, | polyurethane |
| | carboxyl, or mixture | |
| | thereof | |
| amine | carboxyl | polyamide |
| epoxy | hydroxyl | polyepoxy |
| carboxyl | hydroxyl | polyester |

The labels G1 and G2 may be reversed. In some polymerizations, a single monomer has a G1 and a G2 group, and a collection of molecules of such a monomer can form a polymer chain. In other polymerizations, one monomer has two G1 groups and another monomer has two G2 groups. The mixture of these two monomers can react to form a polymer.

As used herein, a “solution process” is a process for applying a layer of a material or mixture of materials to a substrate. In a solution process, a solution is formed by dissolving the material or materials in a solvent, then applying a layer of the solution to the substrate, then evaporating the solvent. The layer of solution may be formed by any method, including, for example, spin coating, slot-die coating, micro-dispensing, or an ink jet method.

When a ratio is said herein to be X:1 or greater, it is meant that the ratio is Y:1, where Y is greater than or equal to X. For example, if a ratio is said to be 3:1 or greater, that ratio may be 3:1 or 5:1 or 100:1 but may not be 2:1. Similarly, when a ratio is said herein to be W:1 or less, it is meant that the ratio is Z:1, where Z is less than or equal to W. For example, if a ratio is said to be 15:1 or less, that ratio may be 15:1 or 10:1 or 0.1:1 but may not be 20:1.

The composition of the present invention comprises a polymer. Any of a wide variety of polymer compositions may be used. Some preferred types of polymers are vinyl polymers, polyurethanes, polyamides, polycarbonates, polyepoxies, and conjugated polymers. That is, the polymer preferably contains the reaction products of carbon-carbon double bonds, or urethane linkages, or urea linkages, or ester linkages, or amide linkages, or —OCH₂CH(OH)CH₂— linkages; or sp² hybridized carbon-carbon single bonds; more preferably reaction products of carbon-carbon double bonds or sp² hybridized carbon-carbon single bonds; more preferably reaction products of carbon-carbon double bonds.

The polymer contains the structure (S1):

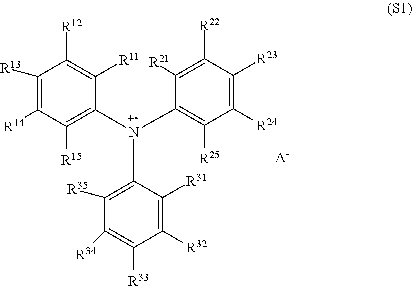

The structure (S1) is referred to herein as a triaryl aminium radical cation.

The groups R¹¹, R¹², R¹³, R¹⁴, R¹⁵, R²¹, R²², R²³, R²⁴, R²⁵, R³¹, R³², R³³, R³⁴, and R³⁵ are called herein the “S1R groups.” Each of the S1R groups is independently selected from hydrogen, deuterium, halogens, amine groups, hydroxyl groups, sulfonate groups, nitro groups, and organic groups. One or more of the S1R groups is covalently bound to the polymer.

In some embodiments (herein “ring embodiments”), two or more S1R groups are covalently bound to each other to form a ring structure. Among ring embodiments, preferred are those in which either (i) the pair of S1R groups that are bound to each other are adjacent to each other on a single aromatic ring or (ii) the pair of S1R groups is selected from the following: R³¹ to R²⁵; R¹⁵ to R³⁵; and R¹¹ to R²¹. In case (ii), it is possible that the two bonded S1R groups are combined in such a way that a single atom of the combined S1R group is bonded to two of the aromatic rings shown in structure (S1). Another possibility in case (ii) is that combined S1R group has no atoms; such an S1R group would consist of a bond that connected a carbon atom on one of the aromatic rings shown in structure (S1) to a carbon atom on one of the other aromatic rings shown in structure (S1).

Each aminium radical cation S1 group is associated with an anion A⁻. The anion A⁻ may be any composition. The anion A⁻ may be located in any of a variety of places. For example, A⁻ may be a group that is covalently attached to the polymer that contains structure (S1), or A⁻ may be a separate atom or molecule. Preferably, A⁻ is not covalently bound to the polymer that contains structure (S1). A⁻ may be an atomic anion or a molecular anion. Molecular anion may be a dimer or an oligomer or a polymer or a molecule that is not a dimer or an oligomer or a polymer. Preferably, A⁻ is a molecular anion that is not a polymer.

Preferred anions A⁻ are BF₄⁻, PF₆⁻, SbF₆⁻, AsF₆⁻, ClO₄⁻, anions of structure SA, anions of structure MA, and mixtures thereof. Structure SA is

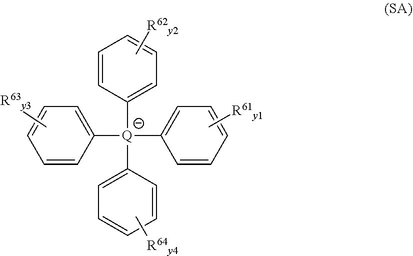

where Q is B, Al, or Ga, preferably B, and where each of y1, y2, y3, and y4 is independently 0 to 5, meaning that there are zero to 5 R groups (i.e, R⁶¹ or R⁶² or R⁶³ or R⁶⁴) present on each of the four aromatic rings appearing in structure (SA). Any pair of the R groups in structure (SA) may be the same as each other or different from each other. Each R group in structure (SA) is independently selected from hydrogen, deuterium, a halogen, an alkyl, or a halogen-substituted alkyl. Any two R groups in structure (SA) may be bonded together to form a ring structure. Among anions with structure SA, preferred are those with one or more R groups selected from deuterium, fluorine, and trifluormethyl.

Structure MA is

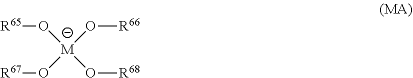

where M is B, Al, or Ga, preferably Al; and where each of R⁶⁵, R⁶⁶, R⁶⁷, and R⁶⁸ is independently alkyl, aryl, fluoroaryl, or fluoroalkyl. Preferably structure MA has 50 or fewer non-hydrogen atoms. Preferred anions are BF₄⁻ and anions of structure (SA); more preferably anions of structure (SA).

In some suitable embodiments, A has the structure (SA) where one R⁶¹ group or one R⁶² group or one R⁶³ group or one R⁶⁴ group or a combination thereof has the structure (SA2):

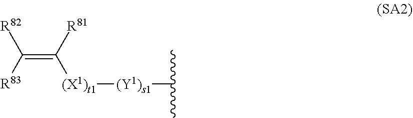

where each of R⁸¹, R⁸², and R⁸³ is hydrogen or a hydrocarbon group having 1 to 20 carbon atoms; where X¹ is an alkylene group having 1 to 20 carbon atoms; where Y¹ is an allylene group having 6 to 20 carbon atoms; where s1 is 0 or 1; where t1 is 0 or 1; and where (t1+s1) is 1 or 2. In structure (SA2), the Y¹ group farthest to the right is bonded to a carbon atom in an aromatic ring shown if structure (SA), which in turn is bonded to Q. When structure (SA2) is present, the preferred Q is boron.

Preferably the polymer is a vinyl polymer. When the polymer is a vinyl polymer, one or more of the S1R groups contains one or more residues of a reaction of a carbon-carbon double bond with other carbon-carbon double bonds in a vinyl polymerization reaction. Also contemplated are embodiments in which the polymer is the result of a polymerization reaction that includes a reaction of complementary reactive groups G1 and G2; in such embodiments, one of the following situations occurs:
(a) one or more of the S1R groups contains a residue of G1 after it has reacted with G2, and a different one of the S1R groups on the same structure (S1) contains a residue of G2 after it has reacted with G1, or
(b) some of the polymerized units, two or more of the S1R groups each contain a residue of G1 after it has reacted with G2, and on other of the polymerized units, two or more of the S1R groups each contain a residue of G2 after it has reacted with G1.

Preferably, two or more of the S1R groups are hydrogen; more preferably 4 or more; more preferably 6 or more; more preferably 8 or more; more preferably 10 or more. Among S1R groups that are not hydrogen, preferred are organic groups having 50 or fewer carbon atoms. Preferably, one or more of R¹¹, R¹², R¹³, R¹⁴, and R¹⁵ is an organic group having one or more aromatic groups. Preferably, one or more of R²¹, R²², R²³, R²⁴, and R²⁵ is an organic group having one or more aromatic groups. Preferably, one or more of R³¹, R³², R³³, R³⁴, and R³⁵ is an organic group having one or more aromatic groups. Preferably, each of R¹¹, R¹², R¹³, R¹⁴, and R¹⁵ is either hydrogen or a hydrocarbyl group. Preferably, each of R²¹, R²², R²³, R²⁴, and R²⁵ is either hydrogen or a hydrocarbyl group. Preferably, each of R³¹, R³², R³³, R³⁴, and R³⁵ is either hydrogen or an organic group containing one or more heteroatoms; preferred heteroatom is nitrogen; preferably the heteroatom is part of a heteroaromatic groups. Preferably, any S1R group that is not hydrogen has 50 or fewer atoms other than hydrogen.

Among S1R groups that are not hydrogen, preferred organic groups are the following. The point of attachment to structure (S1) is shown by the jagged line symbol [symbol] . Where a single group has two points of attachment, that group attaches to two adjacent carbon atoms on one of the aromatic rings in structure (S1).

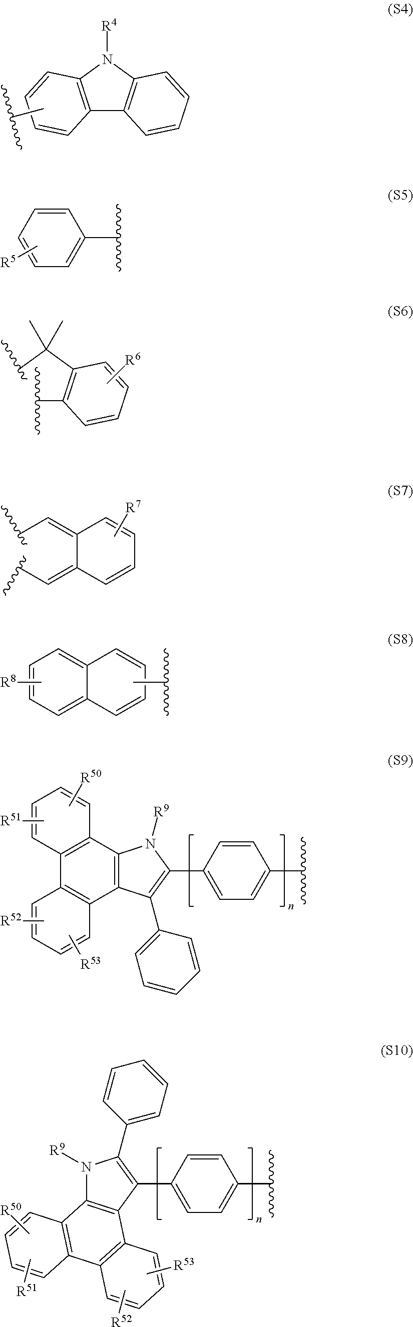

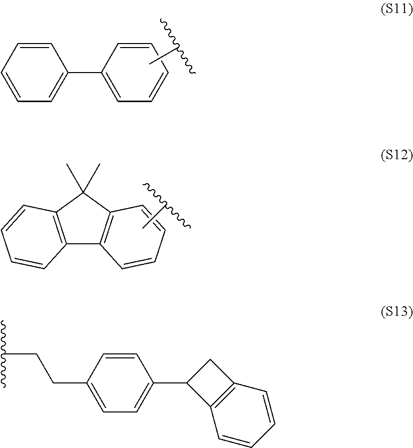

Wherein each of R⁴, R⁵, R⁶, R⁷, R⁸, R⁹, R⁵⁰, R⁵¹, R⁵², and R⁵³ is a hydrogen or an organic group. Preferably R⁵ is a hydrogen, an alkyl group, or an organic group containing an aromatic ring. One preferred R⁵ is a structure (S14), where the portion in brackets is the residue of a reaction of a carbon-carbon double bond with another carbon-carbon double bonds in a vinyl polymerization reaction. Preferably n is 1 or 2. Preferred organic groups have 50 or fewer atoms other than hydrogen. Structure (S14) is the following:

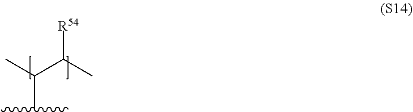

where R⁵⁴ is hydrogen or an alkyl group, preferably hydrogen or a C₁ to C₄ alkyl group, preferably hydrogen or methyl, more preferably hydrogen.

Preferably R⁴ is an alkyl group (preferably methyl) or a group of structure (S5). In some embodiments, when R⁴ has structure S5, R⁵ is structure (S14), and R¹⁴ is hydrogen. Preferably R⁶ is hydrogen. Preferably R⁷ is hydrogen. Preferably R⁸ is hydrogen. In structures (S9) and (S10), each of R⁹, R⁵⁰, R⁵¹, R⁵², and R⁵³ is preferably hydrogen, an alkyl group, or a group of structure (S5). In structures (S9) and (S10), n is an integer from 0 to 10; preferably 0 to 2.

Preferably, R¹¹, R¹², R¹⁴, and R¹⁵ are all hydrogens. Preferably, R²¹, R²², R²⁴, and R²⁵ are all hydrogens. Preferably, R³¹, R³⁴, and R³⁵ are all hydrogens. More preferred are embodiments (herein called “(I)” embodiments, in which R¹¹, R¹², R¹⁴, R¹⁵, R²¹, R²², R²⁴, R²⁵, R³¹, R³⁴ and R³⁵ are all hydrogens.

Also preferred are embodiments (herein called “(II)” embodiments), in which R³² and R³³ together have structure (S6), preferably where R⁶ is hydrogen.

Among I embodiments, preferred are those that are also II embodiments.

Some preferred embodiments, labeled herein A embodiments, B embodiments, and C embodiments, are as follows.

In A embodiments, R²³ is structure (S4), preferably with R⁴ having structure (S5), preferably with R⁵ having structure (S14), preferably with R⁵⁴ being hydrogen. Among A embodiments, preferably R¹³ is structure (S5), preferably with R⁵ being a hydrogen. Preferred A embodiments are also I embodiments.

In B embodiments, R²³ is structure (S5), preferably with R⁵ having structure (S14), preferably with R⁵⁴ being hydrogen. Among B embodiments, preferably R¹³ is structure (S4), preferably with R⁴ having structure (S5), preferably with R⁵ being a hydrogen. Preferred B embodiments are also I embodiments.

In C embodiments, R²³ is structure (S5), preferably with R⁵ having structure (S14), preferably with R⁵⁴ being hydrogen. Among C embodiments, preferably R¹³ is structure (S5), preferably with R⁵ being a hydrogen. Preferred C embodiments are also I embodiments.

In some preferred embodiments, one or more of R¹¹, R¹², R¹³, R¹⁴, R¹⁵, R²¹, R²², R²³, R²⁴, R²⁵, R³¹, R³², R³³, R³⁴, and R³⁵ is selected from substituted or unsubstituted phenyl, substituted or unsubstituted carbazolyl, substituted or unsubstituted indolyl, substituted or unsubstituted fluorenyl, or substituted or unsubstituted biphenyl.

In some preferred embodiments, structure (S1) has the structure (S201):

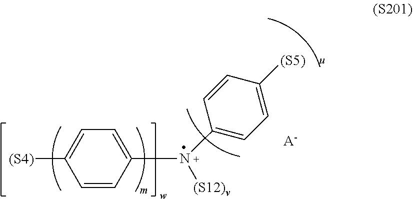

Where A, S4, S5, and S12 are defined above. The index m is 0 or 1. The index u is 0 to 3, and it denotes that 0 to 3 groups in curved brackets with subscript u may be attached to the nitrogen atom. Each S5 group has an R⁵ group attached to it. In these embodiments, R⁵ is H or styrenyl or vinyl. If u is 2 or 3, each of the various R⁸ groups is chosen independently of the other R⁸ groups. The index v is 0 to 3, and it denotes that 0 to 3 (S12) groups may be attached to the nitrogen atom. The index w is 0 to 3, and it denotes that 0 to 3 of the groups in square brackets may be attached to the nitrogen atom. Each S4 group has an R⁴ group attached to it. In these embodiments, R⁴ is H or styrenyl or vinyl. If w is 2 or 3, each of the various R⁴ groups is chosen independently of the other R⁴ groups. Also, (u+v+w)=3.

Preferably, the polymer contains, in addition to structure (S1), one or more triaryl amine structures having structure (S2):

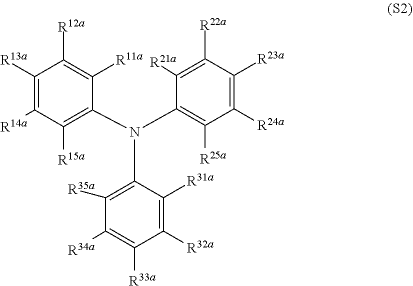

The suitable and preferred structures for R^{11a}, R^{12a}, R^{13a}, R^{14a}, R^{15a}, R^{21a}, R^{22a}, R^{23a}, R^{24a}, R^{25a}, R^{31a}, R^{32a}, R^{33a}, R^{34a}, and R^{35a} are the same as those described above for R¹¹, R¹², R¹³, R¹⁴, R¹⁵, R²¹, R²², R²³, R²⁴, R²⁵, R³¹, R³², R³³, R³⁴, and R³⁵ in structure (S2). The groups R^{11a}, R^{12a}, R^{13a}, R^{14a}, R^{15a}, R^{21a}, R^{22a}, R^{23a}, R^{24a}, R^{25a}, R^{31a}, R^{32a}, R^{33a}, R^{34a}, and R^{35a} are known herein as the S2R groups. Each S2R group has a label of format R^{ija} and is said herein to correspond to the S1R group having label of format R^{ij} and having the same values of i and j. For example, R^{31a} in S2 is said to correspond to R³¹ in S1. Each S2R group may or may not be the same as the corresponding S1R group. In some embodiments, one or more S2R group(s) is (are) different from its (their) corresponding S1R group(s). Preferably, the polymer contains one or more S2 groups in which every S2R group is the same as the corresponding S1R group.

The following is a list (“list A”) of suitable S2 structures. It is noted that each structure in list A contains a nitrogen atom attached to three aromatic rings (the “triaryl” nitrogen). It is contemplated that the triaryl nitrogen atom in each structure in list A could be oxidized to form an aminium radical cation, thus forming a suitable structure that corresponds to the S2 structure shown in list A. List A is as follows:

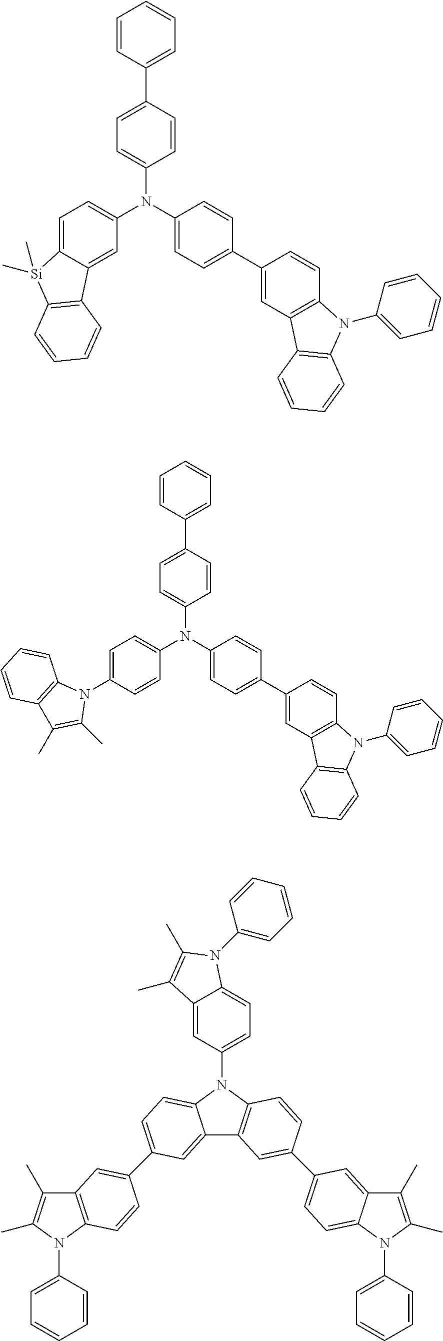

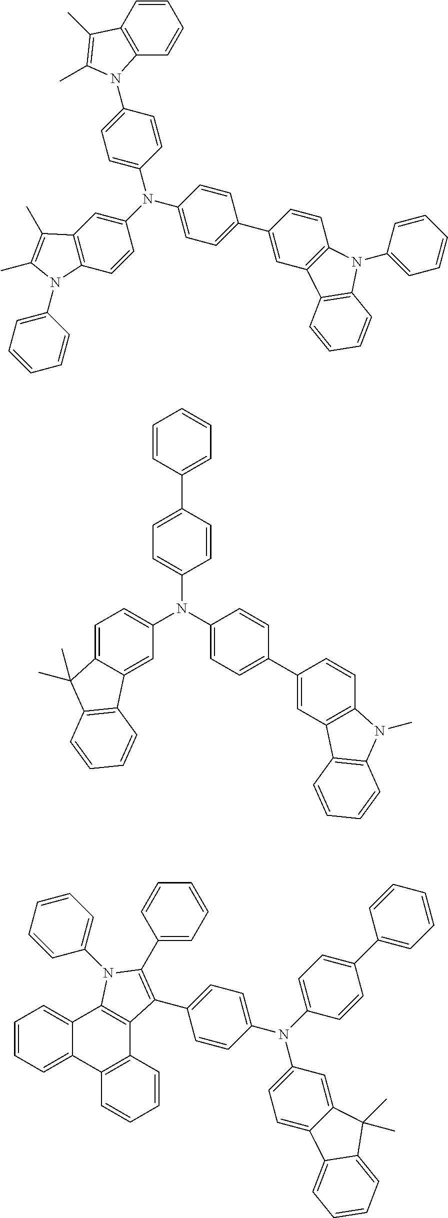

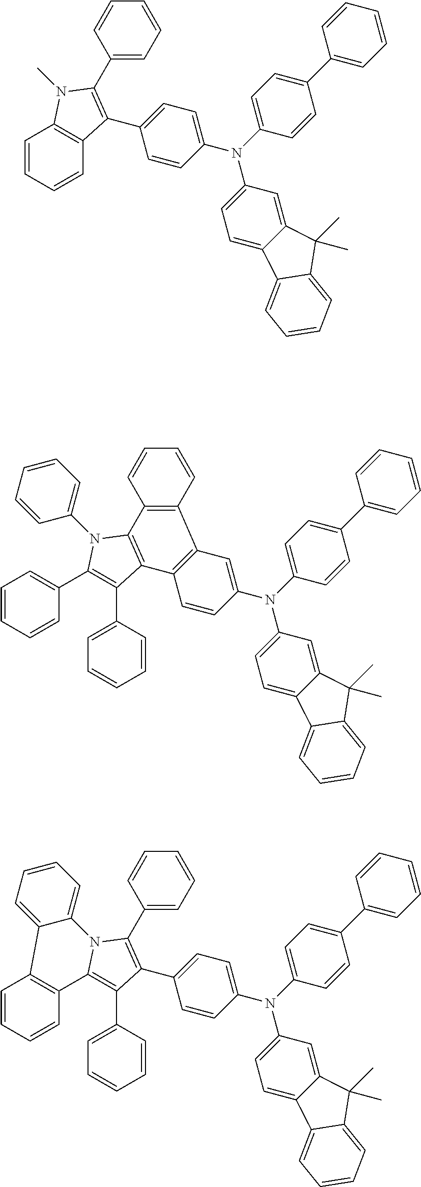 [SMILES: Cn1c(-c2ccccc2)c(-c2ccc(N(c3ccc(-c4ccccc4)cc3)c3ccc4c(c3)C(C)(C)c3ccccc3-4)cc2)c2ccccc21 ; CC1(C)c2ccccc2-c2ccc(N(c3ccc(-c4ccccc4)cc3)c3ccc4c(c3)c3ccccc3c3c4c(-c4ccccc4)c(-c4ccccc4)n3-c3ccccc3)cc21 ; CC1(C)c2ccccc2-c2ccc(N(c3ccc(-c4ccccc4)cc3)c3ccc(-c4c(-c5ccccc5)c5c6ccccc6c6ccccc6n5c4-c4ccccc4)cc3)cc21]

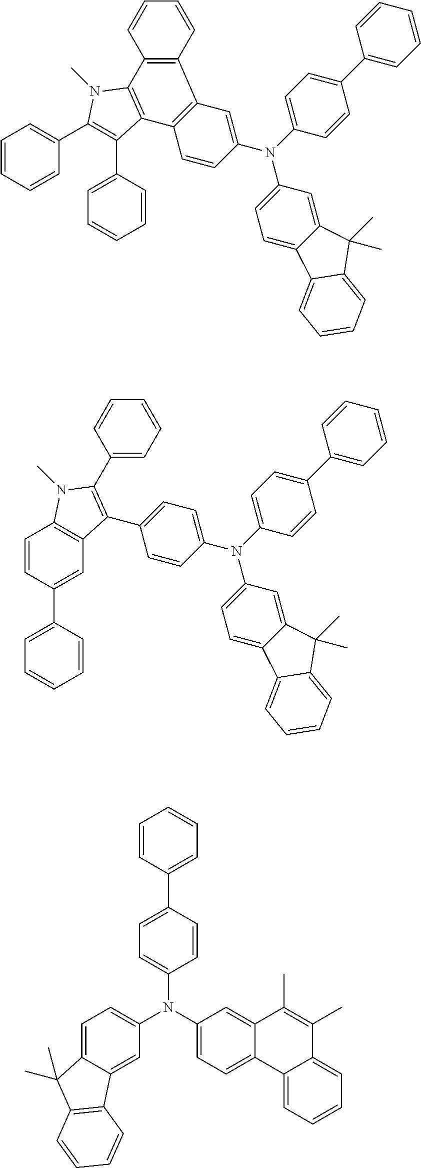

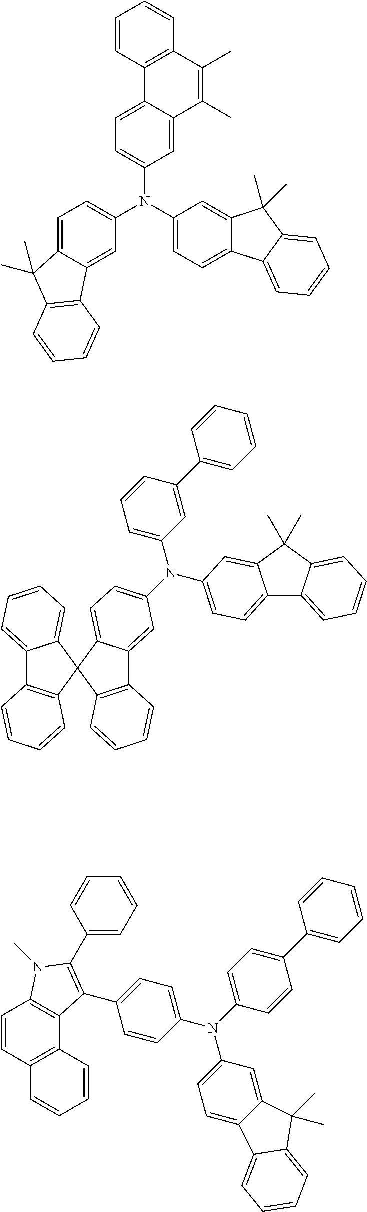 [SMILES: Cc1c(C)c2cc(N(c3ccc4c(c3)-c3ccccc3C4(C)C)c3ccc4c(c3)C(C)(C)c3ccccc3-4)ccc2c2ccccc12 ; CC1(C)c2ccccc2-c2ccc(N(c3cccc(-c4ccccc4)c3)c3ccc4c(c3)-c3ccccc3C43c4ccccc4-c4ccccc43)cc21 ; Cn1c(-c2ccccc2)c(-c2ccc(N(c3ccc(-c4ccccc4)cc3)c3ccc4c(c3)C(C)(C)c3ccccc3-4)cc2)c2c3ccccc3ccc21]

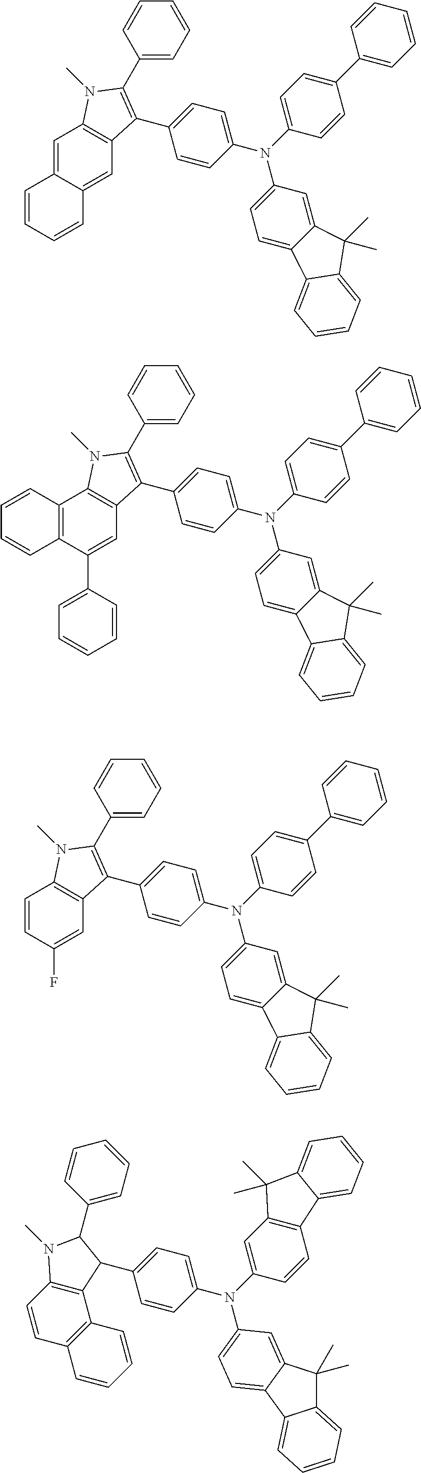 [SMILES: Cn1c(-c2ccccc2)c(-c2ccc(N(c3ccc(-c4ccccc4)cc3)c3ccc4c(c3)C(C)(C)c3ccccc3-4)cc2)c2cc3ccccc3cc21 ; Cn1c(-c2ccccc2)c(-c2ccc(N(c3ccc(-c4ccccc4)cc3)c3ccc4c(c3)C(C)(C)c3ccccc3-4)cc2)c2cc(-c3ccccc3)c3ccccc3c21 ; Cn1c(-c2ccccc2)c(-c2ccc(N(c3ccc(-c4ccccc4)cc3)c3ccc4c(c3)C(C)(C)c3ccccc3-4)cc2)c2cc(F)ccc21 ; CN1c2ccc3ccccc3c2C(c2ccc(N(c3ccc4c(c3)C(C)(C)c3ccccc3-4)c3ccc4c(c3)C(C)(C)c3ccccc3-4)cc2)C1c1ccccc1]

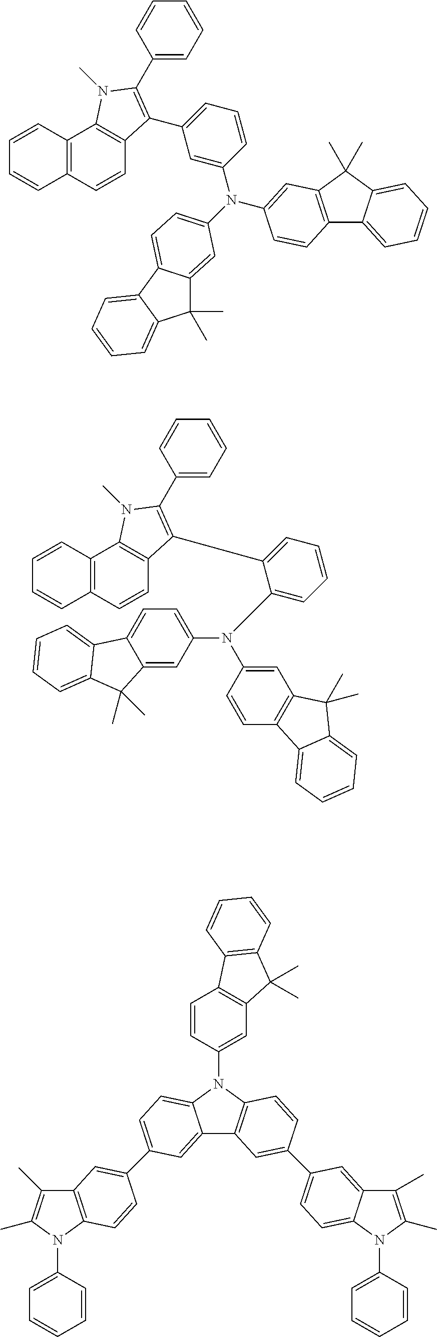

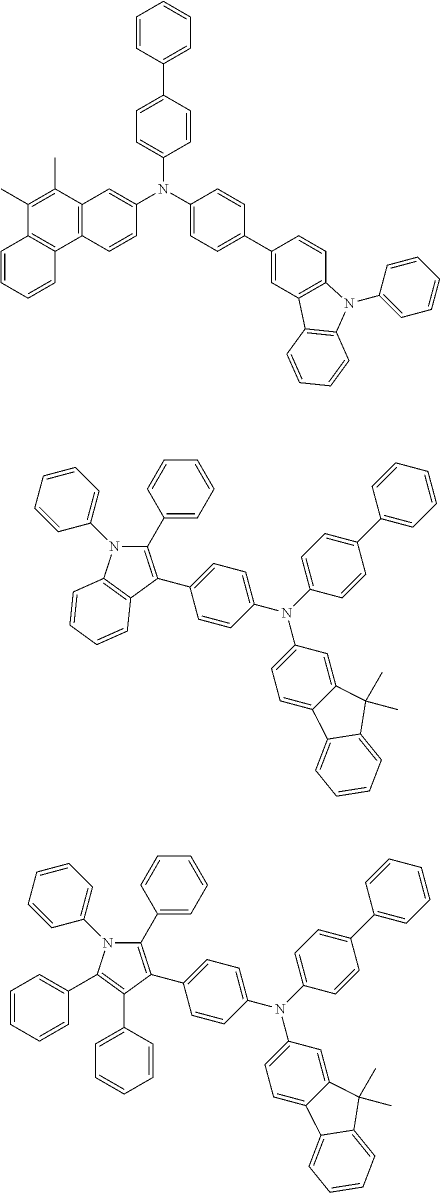 [SMILES: Cc1c(C)c2cc(N(c3ccc(-c4ccccc4)cc3)c3ccc(-c4ccc5c(c4)c4ccccc4n5-c4ccccc4)cc3)ccc2c2ccccc12 ; CC1(C)c2ccccc2-c2ccc(N(c3ccc(-c4ccccc4)cc3)c3ccc(-c4c(-c5ccccc5)n(-c5ccccc5)c5ccccc45)cc3)cc21 ; CC1(C)c2ccccc2-c2ccc(N(c3ccc(-c4ccccc4)cc3)c3ccc(-c4c(-c5ccccc5)c(-c5ccccc5)n(-c5ccccc5)c4-c4ccccc4)cc3)cc21]

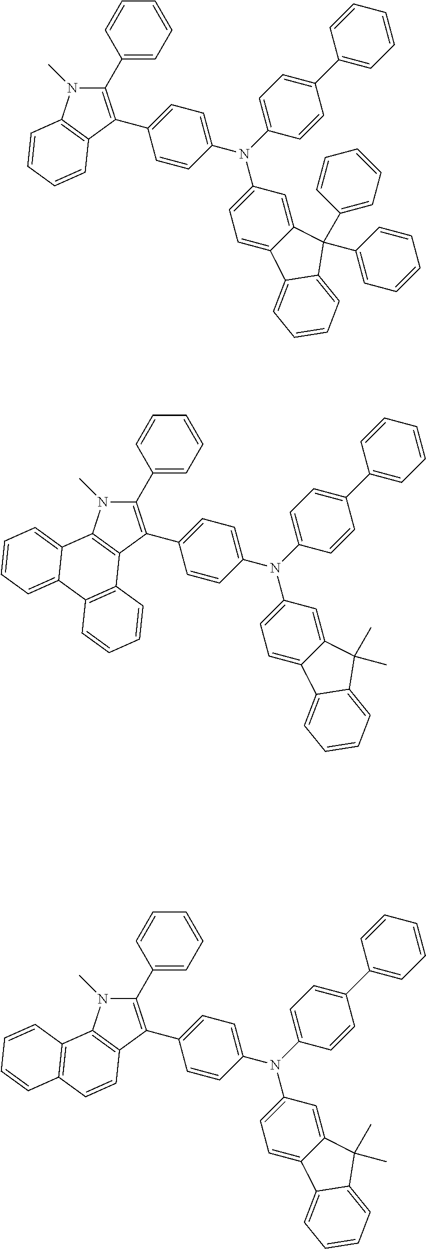 [SMILES: Cn1c(-c2ccccc2)c(-c2ccc(N(c3ccc(-c4ccccc4)cc3)c3ccc4c(c3)C(c3ccccc3)(c3ccccc3)c3ccccc3-4)cc2)c2ccccc21 ; Cn1c(-c2ccccc2)c(-c2ccc(N(c3ccc(-c4ccccc4)cc3)c3ccc4c(c3)C(C)(C)c3ccccc3-4)cc2)c2c3ccccc3c3ccccc3c21 ; Cn1c(-c2ccccc2)c(-c2ccc(N(c3ccc(-c4ccccc4)cc3)c3ccc4c(c3)C(C)(C)c3ccccc3-4)cc2)c2ccc3ccccc3c21]

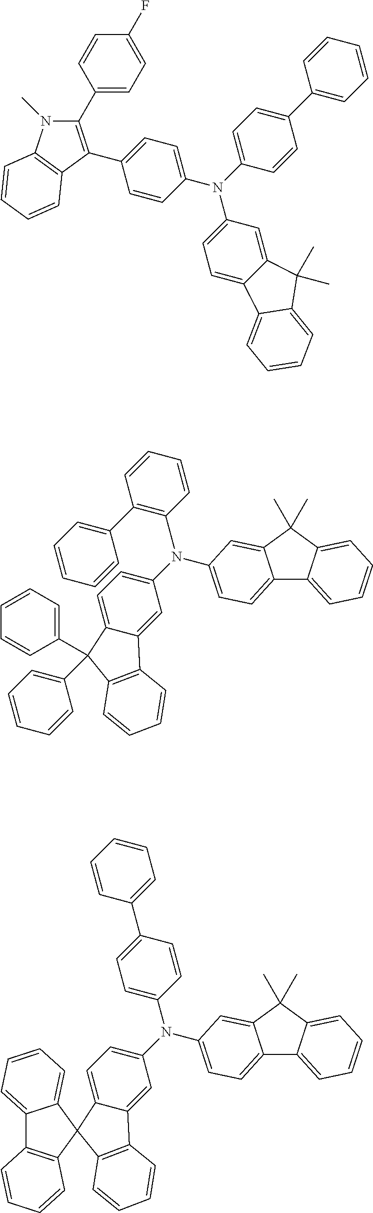 [SMILES: Cn1c(-c2ccc(F)cc2)c(-c2ccc(N(c3ccc(-c4ccccc4)cc3)c3ccc4c(c3)C(C)(C)c3ccccc3-4)cc2)c2ccccc21 ; CC1(C)c2ccccc2-c2ccc(N(c3ccc4c(c3)-c3ccccc3C4(c3ccccc3)c3ccccc3)c3ccccc3-c3ccccc3)cc21 ; CC1(C)c2ccccc2-c2ccc(N(c3ccc(-c4ccccc4)cc3)c3ccc4c(c3)-c3ccccc3C43c4ccccc4-c4ccccc43)cc21]

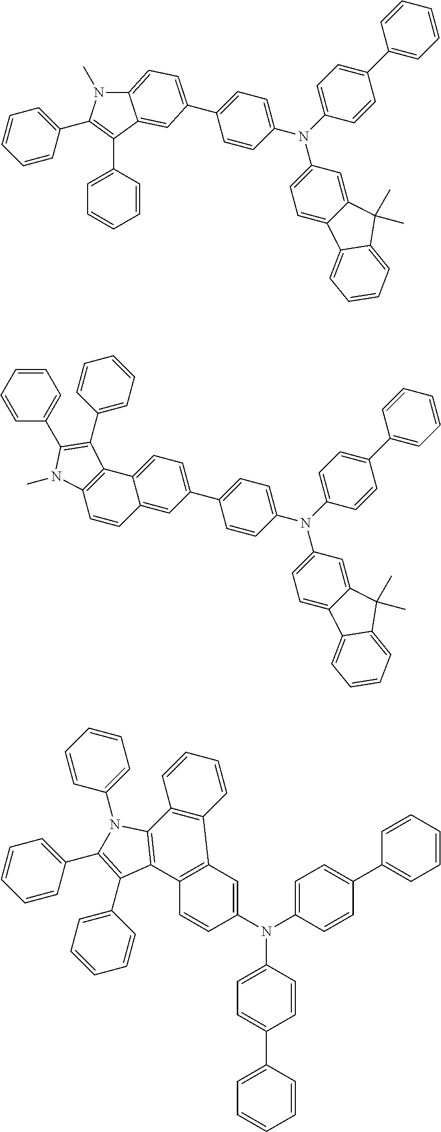 [SMILES: Cn1c(-c2ccccc2)c(-c2ccccc2)c2cc(-c3ccc(N(c4ccc(-c5ccccc5)cc4)c4ccc5c(c4)C(C)(C)c4ccccc4-5)cc3)ccc21 ; Cn1c(-c2ccccc2)c(-c2ccccc2)c2c3ccc(-c4ccc(N(c5ccc(-c6ccccc6)cc5)c5ccc6c(c5)C(C)(C)c5ccccc5-6)cc4)cc3ccc21 ; c1ccc(-c2ccc(N(c3ccc(-c4ccccc4)cc3)c3ccc4c(c3)c3ccccc3c3c4c(-c4ccccc4)c(-c4ccccc4)n3-c3ccccc3)cc2)cc1]

Preferably, in the polymer of the present invention, the mole ratio of S2 groups to S1 groups is 999:1 or lower; more preferably 500:1 or lower; more preferably 99:1 or lower; more preferably 50:1 or lower; more preferably 20:1 or lower. Preferably, in the polymer of the present invention, the mole ratio of S2 groups to S1 groups is 0.001: or greater; more preferably 2:1 or greater; more preferably 3.5:1 or greater; more preferably 5.5:1 or greater.

The polymer of the present invention preferably has number-average molecular weight of 2,500 Da or higher; more preferably 5,000 Da or higher, more preferably 10,000 Da or higher; more preferably 20,000 Da or higher; more preferably 40,000 Da or higher; more preferably 60,000 Da or higher. The polymer of the present invention preferably has number-average molecular weight of 500,000 Da or lower; more preferably 300,000 Da or lower; more preferably 150,000 Da or lower.

In some embodiments, the composition of the present invention contains both the polymer described above and one or more additional polymers that contains no structure S1. In some embodiments, the composition contains one or more polymers that contains no structure S1 and no structure S2. If any such additional polymer is present, preferably the additional polymer is the same type of polymer as the polymer that contains structure S1.

Preferably, the polymer of the present invention is at least 99% pure, as measured by liquid chromatography/mass spectrometry (LC/MS) on a solids weight basis, more preferably at least 99.5%, more preferably at least 99.7%. Preferably, the formulation of this invention contains no more than 10 ppm by weight of metals, preferably no more than 5 ppm.

The polymer of the present invention may be made by any method. One method is to polymerize one or more monomers that contain a structure S1, optionally along with one or more other monomers. A preferred method is to first polymerize one or more monomers that contains a structure S2, optionally along with one or more other monomers, and then to subject the resulting polymer to a chemical reaction that converts some or all of the structures S2 in the polymer to structures S1. A preferred method of converting some or all of the structures S2 to structures S1 is to react the polymer that contains structures S2 with one or more oxidizing agents. The reaction with an oxidizing agent is contemplated to proceed as shown in reaction X1:

OA^{⊕}X^{⊖}+S2→OA+S2^{⊕}X^{⊖} (X1)

where OA^{⊕} is the oxidizing agent, X^{⊖} is an anion, and S2^{⊕} is the same as S1. Preferably, the mole ratio of OA^{⊕} to S2 is 25:1 or lower; more preferably 20:1 or lower; more preferably 15:1 or lower. Preferably, the mole ratio of OA^{⊕} to S2 is 2:1 or greater; more preferably 4:1 or greater; more preferably 8:1 or greater.

Preferred oxidizing agents are compounds containing Ag(I) ions (that is, silver ions with +1 valence) and compounds containing nitrosonium ions. Among compounds containing Ag(I) ions, preferred is Ag(I) tetra(pentafluorophenyl)borate. Among compounds containing nitrosonium ions, preferred is NOBF₄. In some embodiments, OA^{⊕} is an onium compound.

Preferably, the reaction between the polymer and the oxidizing agent is carried out in an organic solvent. When the oxidizing agent is a compound containing a Ag(I) ion, preferred solvents contain one or more aromatic rings; more preferably the aromatic ring has no heteroatom; more preferably the solvent contains one or more heteroatoms that is not located within an aromatic ring; more preferably the solvent is anisole. When the oxidizing agent is a compound containing a nitrosonium ion, preferred solvents contain no aromatic ring; more preferred are non-aromatic solvents that contain one or more heteroatoms; more preferred are acetonitrile, dichloromethane, and mixtures thereof.

The polymer may be made by any polymerization method.

In a preferred polymerization method (“vinyl polymerization”), a first monomer is provided that contains structure S2 and that also contains one or more vinyl groups. Preferred vinyl groups have the structure (S15)

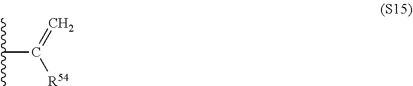

where R⁵⁴ is hydrogen or alkyl, preferably hydrogen or methyl, more preferably hydrogen. Preferably, the attachment point shown in structure S15 attaches to a carbon atom in an aromatic ring. The first monomer may optionally be mixed with additional monomers that contain a vinyl group, and these additional monomers may or may not contain a structure S2 that is different from the S2 group in the first monomer. Preferably, the first monomer contains exactly one vinyl group per molecule. Optionally, one or more of any additional monomers may be monomers that contain two or more vinyl groups per molecule. In vinyl polymerization, the various vinyl groups participate in a polymerization reaction to form a vinyl polymer. The vinyl polymerization may proceed by free-radical polymerization or by one or more other mechanisms; preferably by free-radical polymerization. Preferably, after polymerization, some or all of the S2 groups are converted to S1 groups by an oxidation reaction.

Also contemplated are other polymerization methods different from vinyl polymerization. Preferred among such methods are polymerization methods involving complementary reactive groups G1 and G2 that react with each other (“G1/G2” methods). In some embodiments, a primary monomer is provided that has two or more G1 groups per molecule, and that primary monomer is mixed with a secondary monomer that has two or more G2 groups per molecule, and one or both of the primary and secondary monomer has an S2 group. Then, when the G1 and G2 groups react with each other, a polymer is formed. In other embodiments, a monomer is provided that has a G1 group, a G2 group, and an S2 group. Then, when the G1 and G2 groups react with each other, a polymer is formed. In G1/G2 methods, additional monomers may optionally be present. Preferably, in G1/G2 methods, after polymerization, some or all of the S2 groups are converted to S1 groups by an oxidation reaction.

Preferably, the polymer of the present invention is present as a thin layer on a substrate. The film is preferably formed on a substrate by a solution process, preferably by spin coating or by an ink jet process.

When a solution is made for coating the polymer on a substrate, preferably the solvent has a purity of at least 99.8% by weight, as measured by gas chromatography-mass spectrometry (GC/MS), preferably at least 99.9% by weight. Preferably, solvents have an RED value (relative energy difference (versus polymer) as calculated from Hansen solubility parameter using CHEMCOMP v2.8.50223.1) less than 1.2, more preferably less than 1.0. Preferred solvents include aromatic hydrocarbons and aromatic-aliphatic ethers, preferably those having from six to twenty carbon atoms. Anisole, mesitylene, xylene, and toluene are especially preferred solvents.

Preferably, the thickness of the polymer films produced according to this invention is from 1 nm to 100 microns, preferably at least 10 nm, preferably at least 30 nm, preferably no greater than 10 microns, preferably no greater than 1 micron, preferably no greater than 300 nm.

When the film has been produced by spin coating, the spin-coated film thickness is determined mainly by the solid contents in solution and the spin rate. For example, at a 2000 rpm spin rate, 2, 5, 8, and 10 wt % polymer, formulated solutions result in film thicknesses of 30, 90, 160 and 220 nm, respectively. Preferably the wet film shrinks by 5% or less after baking and annealing.

The composition of the present invention may be used for any purpose. A preferred use for the composition of the present invention is in one or more layers of an organic light-emitting diode (OLED). An OLED contains an anode, an emitting layer, and a cathode. An OLED optionally contains one or more additional layers.

Preferably, an OLED contains the following layers in contact with each other in order as follows: a substrate, an anode layer, optionally one or more hole injection layers, one or more hole transport layers, optionally one or more electron blocking layers, an emitting layer, optionally one or more hole blocking layers, optionally one or more electron transport layers, an electron injection layer, and a cathode.

Preferably, the OLED contains an electron blocking layer

An embodiment of an OLED is shown in FIG. 1. Substrate 1 is coated with an anode layer 2. The anode layer is preferably conductive. The anode layer 2 is in contact with an optional hole injection layer (HIL) 3. The other layers are, in order: a hole transport layer (HTL) 4, an optional electron blocking layer (EBL) 5, the emitting layer 6, an optional hole blocking layer (HBL) 7, an electron transport layer (ETL) 8, an optional electron injection layer (EIL) 9, and a cathode 12. The cathode is preferably conductive. When it is desired that the OLED produce emitted light, a voltage source 10 is connected to the OLED via conductors 11 as shown in FIG. 1. The voltage is preferably applied so that the cathode is at a negative voltage relative to the anode.

A common substrate material is glass. Also suitable are transparent substrates made of substances other than glass, including flexible substrates made of substances other than glass. A preferred anode layer is tin-doped indium oxide (ITO). Preferred hole injection layers contain one or more polymer compositions of the present invention. Preferably the emitting layer comprises one or more hosts and one or more emitters. Preferred hosts are aromatic amines. Preferred emitters are phosphorescent emitters. Preferred electron injection layers comprise one or more organometallic compounds; more preferably one or more metal quinolates; more preferably lithium quinolate. Preferred cathode materials are metals. Also contemplated are embodiments in which in which a transparent layer (not shown in FIG. 1), such as glass, is present on top of the cathode 12; in such embodiments, the substrate 1 may or may not be transparent.

The composition of the present invention will be present in either a hole injection layer (HIL), a hole transport layer (HTL), in both a hole injection layer and a hole transport layer, or in a dual-functional layer (HITL) that functions as both a hole injection layer and a hole transport layer. Preferred are embodiments in which the composition of the present invention is present in an HITL.

Preferably, all the layers that contain the composition of the present invention lie between the anode and the emitting layer.

In some embodiments (herein “gradient” embodiments), the OLED contains a “gradient layer” that is located between the anode and the emitting layer, that contains the composition of the present invention, and that has a concentration of S1 groups that is not uniform throughout the thickness of the layer. As portions of the gradient layer are examined in order from the portion closest to the anode to the portion closest to the emitting layer, the concentration of S1 groups may or may not change monotonically. For example, the concentration of S1 groups may increase monotonically, may decrease monotonically, may show a minimum, may show a maximum, or some combination thereof. The concentration of S1 may be assessed by any measure, including, for example, number of S1 groups per unit of volume, or number of S1 groups per unit of mass of polymer.

In the gradient layer, preferably, the concentration of S1 groups is higher in the portion of the gradient layer nearest the anode than in the portion of the gradient layer nearest the emitting layer. The concentration of S1 groups may vary gradually or in sudden steps or in some other way. Preferably, as portions of the gradient layer are examined in order from the portion closest to the anode to the portion closest to the emitting layer, at each portion the concentration of S1 groups is equal to or less than the concentration of S1 groups in the previous portion. The gradient layer may be constructed by a multi-step process, or the gradient layer may be constructed in some other way that results in the gradient of volume concentration of S1 groups. Preferably, the ratio of the concentration of S1 groups in the portion of the gradient layer nearest the anode to the concentration of S1 groups in the portion of the gradient layer nearest the emitting layer is higher than 1:1; or 1.1:1 or higher; or 1.5:1 or higher; or 2:1 or higher; or 5:1 or higher.

In embodiments in which S1 groups and S2 groups are both present in a gradient layer, it is useful to characterize the mole ratio of S2 groups to S1 groups. In the portion of the gradient layer nearest the anode, the mole ratio of S2 groups to S1 groups is defined herein as MRA:1. Preferably MRA is from 1 to 9. In the portion of the gradient layer nearest the emitting layer, the mole ratio of S2 groups to S1 groups is defined as MRE:1. Preferably MRE is from greater than 9 to 999. Preferably MRA is less than MRE. Preferably the ratio of MRA to MRE is less than 1:1; more preferably 0.9:1 or less; more preferably 0.67:1 or less; more preferably 0.5:1 or less; more preferably 0.2:1 or less.

It is contemplated that a benefit of the present invention is that the S1 groups, because they are bonded to a polymer, resist migration. OLEDs are sometimes subjected to elevated temperature, for example during processing and during extended use. Under these conditions, prior to the present invention, an OLED would typically have an HIL and/or an HTL that depended on a non-polymeric dopant to provide its electrical properties. Such non-polymeric dopants can migrate, especially when exposed to elevated temperature, and migration can ruin the functioning of the OLED. In contrast, it is contemplated that an OLED of the present invention will have an HIL and/or HTL that depends on an S1-containing polymer for its electrical properties. Because the polymer will resist migration, an OLED of the present invention is expected to resist the loss of function due to migration that can harm previously known OLEDs.

Preferably, the layer of the OLED that contains the composition of the present invention is resistant to dissolution by solvent (solvent resistance is sometimes referred to as “solvent orthogonality”). Solvent resistance is useful because, after making the layer of the OLED that contains the composition of the present invention, a subsequent layer may be applied to the layer that contains the composition of the present invention. In many cases, the subsequent layer will be applied by a solution process. It is desirable that the solvent in the subsequent solution process does not dissolve or significantly degrade the layer that contains the composition of the present invention. Solvent resistance is assessed using the “strip test” described in the Examples below.

When the composition of the present invention is present in an HIL, preferably the HIL layer will be formed by a solution process. A subsequent layer may be applied to the HIL; the subsequent layer is typically an HTL. The HTL may be applied, for example, by an evaporation process (usually used when the HTL consists of small molecules and does not contain polymer) or a solution process (usually used when the HTL contains one or more polymer). If the HTL is applied by a solution process, preferably the HIL is resistant to the solvents used in the solution process for applying the HTL.

When the composition of the present invention is present in an HTL, preferably the HTL layer will be formed by a solution process. A subsequent layer may be applied to the HTL; the subsequent layer is typically an emitting layer. The emitting layer may be applied, for example, by an evaporation process (usually used when the emitting layer consists of small molecules and does not contain polymer) or a solution process (usually used when the emitting layer contains one or more polymers). If the emitting layer is applied by a solution process, preferably the HTL is resistant to dissolution in the solvent used in the solution process for applying the emitting layer.

When the composition of the present invention is present in an HITL, it is contemplated that the HITL is in contact with an optional additional hole injection layer or the anode on one side and the emitting layer or an optional electron blocking layer on the other side. When an HITL is used, any additional HIL or HTL is not necessarily present in the OLED.

When a layer containing the composition of the present invention is applied to a substrate using a solution process, it is preferred that the solution process be performed as follows. Preferably, a solution is formed that contains a polymer of the present invention dissolved in a solvent. Preferably, then a layer of the solution is applied to a substrate (the substrate is preferably either the anode or a previous layer of an OLED), and the solvent is evaporated or allowed to evaporate to make a thin film. It is preferred that the thin film is then heated to a temperature of 170° C. or above, more preferably 180° C. or above; more preferably 200° C. or above.

Preferably, the duration of the exposure to hot atmosphere is 2 minutes or more; more preferably 5 minutes or more. Preferably the atmosphere is inert; more preferably the atmosphere contains 1% or less by weight oxygen gas; more preferably the atmosphere contains 99% or more nitrogen by weight.

The following are examples of the present invention.

### PREPARATIVE EXAMPLE 1: SUMMARY OF SYNTHESIS OF MONOMER S101

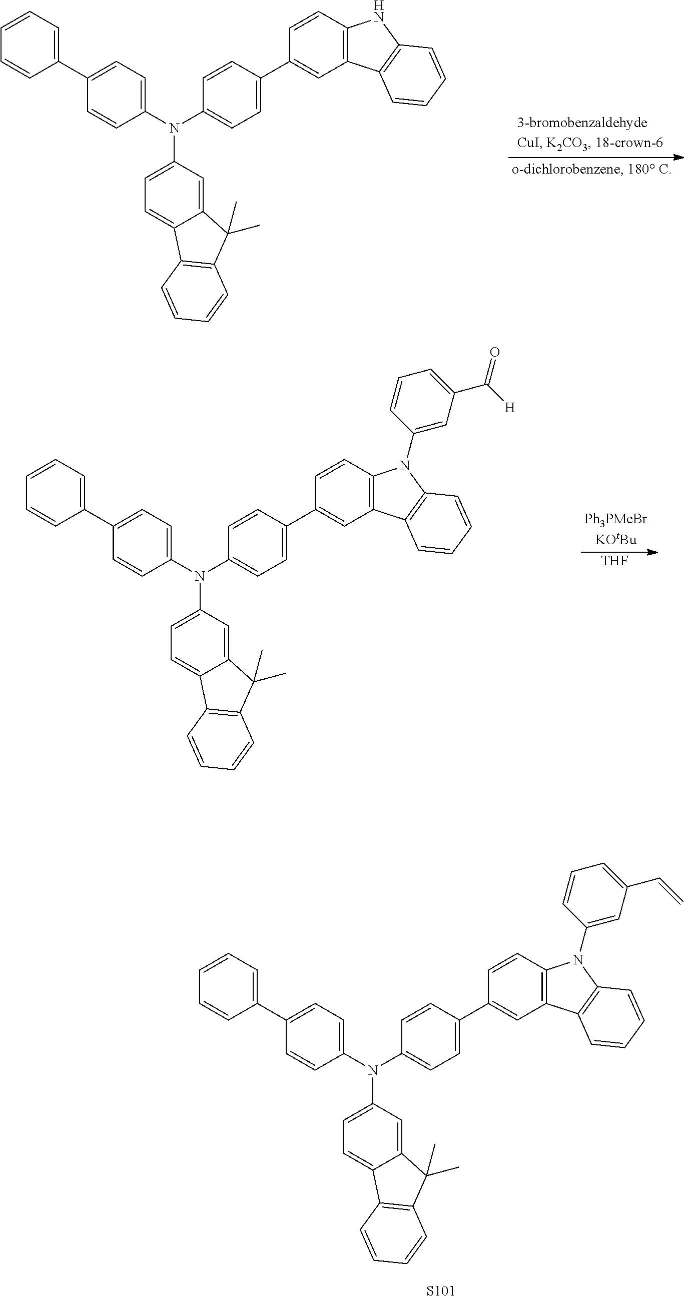 [SMILES: CC1(C)c2ccccc2-c2ccc(N(c3ccc(-c4ccccc4)cc3)c3ccc(-c4ccc5[nH]c6ccccc6c5c4)cc3)cc21 ; CC1(C)c2ccccc2-c2ccc(N(c3ccc(-c4ccccc4)cc3)c3ccc(-c4ccc5c(c4)c4ccccc4n5-c4cccc(C=O)c4)cc3)cc21 ; "THF" ; C=Cc1cccc(-n2c3ccccc3c3cc(-c4ccc(N(c5ccc(-c6ccccc6)cc5)c5ccc6c(c5)C(C)(C)c5ccccc5-6)cc4)ccc32)c1 ; "S101"]

### PREPARATIVE EXAMPLE 2: SYNTHESIS OF 3-(3-(4-([1,1′-BIPHENYL]-4-YL(9,9-DIMETHYL-9H-FLUOREN-2-YL)AMINO)PHENYL)-9H-CARBAZOL-9-YL)BENZALDEHYDE

A round bottom flask was charged with carbazole (9.10 g, 15.1 mmol, 1.0 equiv), 3-bromobenzaldehyde (2.11 mL, 18.1 mmol, 1.2 equiv), CuI (0.575 g, 3.02 mmol, 0.2 equiv), potassium carbonate (6.26 g, 45.3 mmol, 3.0 equiv), and 18-crown-6 (399 mg, 10 mol %). The flask was flushed with nitrogen and connected to a reflux condenser. 55 mL of dry, degassed, 1,2-dichlorobenzene was added, and the mixture was heated to 180° C. overnight. Only partial conversion was noted after 14 hours. An additional 2.1 mL of 3-bromobenzaldehyde was added, and heated continuously for another 24 hours.

The solution was cooled and filtered to remove solids. The filtrate was concentrated and adsorbed onto silica for purification by chromatography (0 to 60% dichloromethane in hexanes), which delivered product as a pale yellow solid (8.15 g, 74%). ¹H NMR (500 MHz, CDCl₃) δ 10.13 (s, 1H), 8.39-8.32 (m, 1H), 8.20 (dd, J=7.8, 1.0 Hz, 1H), 8.13 (t, J=1.9 Hz, 1H), 7.99 (d, J=7.5 Hz, 1H), 7.91-7.86 (m, 1H), 7.80 (t, J=7.7 Hz, 1H), 7.70-7.58 (m, 7H), 7.56-7.50 (m, 2H), 7.47-7.37 (m, 6H), 7.36-7.22 (m, 9H), 7.14 (ddd, J=8.2, 2.1, 0.7 Hz, 1H), 1.46 (s, 6H). ¹³C NMR (126 MHz, CDCl₃) δ 191.24, 155.15, 153.57, 147.22, 146.99, 146.60, 140.93, 140.60, 139.75, 138.93, 138.84, 138.17, 136.07, 135.13, 134.42, 133.53, 132.74, 130.75, 128.75, 128.49, 127.97, 127.79, 127.58, 126.97, 126.82, 126.64, 126.51, 126.36, 125.36, 124.47, 124.20, 123.94, 123.77, 123.60, 122.47, 120.68, 120.60, 120.54, 119.45, 118.88, 118.48, 109.71, 109.58, 46.88, 27.12.

### PREPARATIVE EXAMPLE 3: SYNTHESIS OF N-([1,1′-BIPHENYL]-4-YL)-9,9-DIMETHYL-N-(4-(9-(3-VINYLPHENYL)-9H-CARBAZOL-3-YL)PHENYL)-9H-FLUOREN-2-AMINE (S101)

Under a blanket of nitrogen, a round bottom flask was charged with methyltriphenylphosphonium bromide (14.14 g, 39.58 mmol, 2.00 equiv) and 80 mL dry THF. Potassium tert-butoxide (5.55 g, 49.48 mmol, 2.50 equiv) was added in once portion, and the mixture stirred for 15 minutes. Aldehyde from Preparative Example 2 (13.99 g, 19.79 mmol, 1.00 equiv) was added in 8 mL dry THF. The slurry stirred at room temperature overnight. The solution was diluted with dichloromethane, and filtered through a plug of silica. The pad was rinsed with several portions of dichloromethane.

The filtrate was adsorbed onto silica and purified by chromatography twice (10 to 30% dichloromethane in hexanes), which delivered product as a white solid (9.66 g, 67%) Purity was raised to 99.7% by reverse phase chromatography. ¹H NMR (400 MHz, CDCl₃) δ 8.35 (d, J=1.7 Hz, 1H), 8.18 (dt, J=7.7, 1.0 Hz, 1H), 7.68-7.39 (m, 19H), 7.34-7.23 (m, 9H), 7.14 (dd, J=8.1, 2.1 Hz, 1H), 6.79 (dd, J=17.6, 10.9 Hz, 1H), 5.82 (d, J=17.6 Hz, 1H), 5.34 (d, J=10.8 Hz, 1H), 1.45 (s, 6H). ¹³C NMR (101 MHz, CDCl₃) δ 155.13, 153.57, 147.26, 147.03, 146.44, 141.29, 140.61, 140.13, 139.55, 138.95, 137.99, 136.36, 135.98, 135.06, 134.36, 132.96, 130.03, 128.74, 127.97, 127.77, 126.96, 126.79, 126.63, 126.49, 126.31, 126.11, 125.34, 125.16, 124.67, 124.54, 123.90, 123.55, 123.49, 122.46, 120.67, 120.36, 120.06, 119.44, 118.83, 118.33, 115.27, 110.01, 109.90, 46.87, 27.12.

### PREPARATIVE EXAMPLE 4: PROTOCOL FOR RADICAL POLYMERIZATION

In a glovebox, S101 monomer (1.00 equiv) was dissolved in anisole (electronic grade, 0.25 M). The mixture was heated to 70° C., and AIBN solution (0.20 M in toluene, 5 mol %) was injected. The mixture was stirred until complete consumption of monomer, at least 24 hours (2.5 mol % portions of AIBN solution can be added to complete conversion). The polymer was precipitated with methanol (10x volume of anisole) and isolated by filtration. The filtered solid was rinsed with additional portions of methanol. The filtered solid was re-dissolved in anisole and the precipitation/filtration sequence repeated twice more. The isolated solid was placed in a vacuum oven overnight at 50° C. to remove residual solvent.

### PREPARATIVE EXAMPLE 5: MEASUREMENT OF MOLECULAR WEIGHT OF POLYMER

Gel permeation chromatography (GPC) studies were carried out as follows. 2 mg of HTL polymer was dissolved in 1 mL THF. The solution was filtered through a 0.2 μm polytetrafluoroethylene (PTFE) syringe filter and 50 μl of the filtrate was injected onto the GPC system. The following analysis conditions were used: Pump: Waters™ e2695 Separations Modules at a nominal flow rate of 1.0 mL/min; Eluent: Fisher Scientific HPLC grade THF (stabilized); Injector: Waters e2695 Separations Modules; Columns: two 5 μm mixed-C columns from Polymer Laboratories Inc., held at 40° C.; Detector: Shodex RI-201 Differential Refractive Index (DRI) Detector; Calibration: 17 polystyrene standard materials from Polymer Laboratories Inc., fit to a 3rd order polynomial curve over the range of 3,742 kg/mol to 0.58 kg/mol.

| Monomer | Mₙ | M_{w} | M_{z} | M_{z+1} | M_{w}/Mₙ |
|---|---|---|---|---|---|
| S101 | 23,413 | 88,953 | 176,978 | 266,718 | 3.799 |
| | Da | Da | Da | Da | |

### EXAMPLE 6: OXIDATION OF POLYMER

In a glovebox, the HTL polymer as made in Preparative Example 4 was dissolved in anisole (14 mL/g polymer), and oxidizing agent (Ag(I) tetra(pentafluorophenyl)borate, as described in Inorg. Chem. 2012, 51, 2737-2746) was added in a single portion. After stirring for 24 hours at ambient temperature (approximately 23° C., the solution was filtered through a 0.2 μm syringe filter. The material may be used in solution, or the polymer may be precipitated by addition of an excess of methanol. Various polymers were made using various amounts of oxidizing agent, as follows:

| Polymer | Equivalents of oxidizing agent |
|---|---|
| Designation | per equivalent of monomer |
| p(S101)-00 | comparative polymer made in |
| | Preparative Example 4 |
| p(S101)-02 | 0.02 |
| p(S101)-05 | 0.05 |
| p(S101)-10 | 0.10 |

An alternative method that could be used for oxidizing the polymer is as follows. In a glovebox, a round bottom flask could be charged with the HTL polymer and dichloromethane (50 mL per gram polymer). An equivalent amount of acetonitrile would be added slowly, making sure that precipitation of the substrate did not occur. NOBF₄ (0.0642 M in acetonitrile, 0.1 equiv) would be added dropwise, which would turn the solution deep green. The mixture would be allowed to stir open to the ambient glovebox atmosphere for 30 minutes. Solvent would be removed by vacuum pump.

### PREPARATIVE EXAMPLE 7: EXPERIMENTAL PROCEDURES

Preparation of HTL solution formulation: HTL polymer solid powders were directly dissolved into anisole to make a 2 wt % stock solution. The solution was stirred at 80° C. for 5 to 10 min in N₂ for complete dissolving. The resulting formulation solution was filtered through 0.2 μm PTFE syringe filter prior to depositing onto Si wafer.

Preparation of polymer film: Si wafer was pre-treated by UV-ozone for 2 to 4 min prior to use. Several drops of the above filtered formulation solution were deposited onto the pre-treated Si wafer. The thin film was obtained by spin coating at 500 rpm for 5s and then 2000 rpm for 30s. The resulting film was then transferred into the N₂ purging box. The “wet” film was prebaked at 100° C. for 1 min to remove most of residual anisole. Subsequently, the film was thermally cross-linked at temperature between 160° C. and 220° C. for a time between 10 and 30 min (details below).

Strip test on thermally annealed polymer film was performed as follows. The “Initial” thickness of thermally cross-linked HTL film was measured using an M-2000D ellipsometer (J. A. Woollam Co., Inc.). Then, several drops of o-xylene or anisole were added onto the film to form a puddle. After 90 s, the solvent was spun off at 3500 rpm for 30 s. The “Strip” thickness of the film was immediately measured using the ellipsometer. The film was then transferred into the N₂ purging box, followed by post-baking at 100° C. for 1 min to remove any swollen solvent in the film. The “Final” thickness was measured using the ellipsometer. The film thickness was determined using the Cauchy relationship and averaged over 3×3=9 points in a 1 cm×1 cm area. For a fully solvent resistant film, the total film loss (“Final”-“Initial”) after strip test should be <1 nm, preferably <0.5 nm.

### EXAMPLE 8: STRIP TEST USING O-XYLENE

Films were made and stripped as described above. Films were annealed for 20 minutes at 150° C. and 180° C. or for 10 minutes at 205° C. and 220° C. Results were as follows:

o-xylene Film Loss

| | Annealing Times and Temperature | | | |
|---|---|---|---|---|
| | 20 min | 20 min | 10 min | 10 min |
| Polymer | 150° C. | 180° C. | 205° C. | 220° C. |
| p(S101)-00 | 13 nm | 0 | 0 | 0 |
| (comparative) | | | | |
| p(S101)-10 | 19 nm | 0 | 0 | 0 |

Annealing at temperature above 150° C. improves the polymer's resistance to stripping by o-xylene. The inventive polymer p(S101)-10 is resistant to stripping by o-xylene when annealed at 180° C. and above.

### PREPARATIVE EXAMPLE 9: SYNTHESIS OF S102 AND S103

Using methods similar to Preparative Examples 1-4, the following monomers were synthesized:

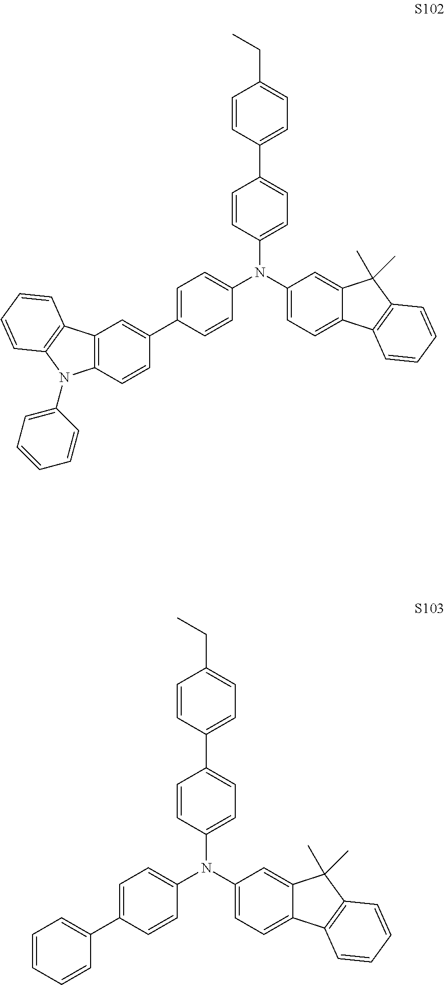 [SMILES: CCc1ccc(-c2ccc(N(c3ccc(-c4ccc5c(c4)c4ccccc4n5-c4ccccc4)cc3)c3ccc4c(c3)C(C)(C)c3ccccc3-4)cc2)cc1 ; CCc1ccc(-c2ccc(N(c3ccc(-c4ccccc4)cc3)c3ccc4c(c3)C(C)(C)c3ccccc3-4)cc2)cc1]

Following the procedure in Preparative Example 4, homopolymers p(S102) and p(S103) were formed. Following the procedures in Preparative Example 6, using 0.10 equivalents of oxidizing agent, partially oxidized polymers having aminium radical cations p(S102)-10 and p(S103)-10 were formed. The oxidizing agent was (Ag(I) tetra(pentafluorophenyl)borate.

### EXAMPLE 10: CALCULATION OF ORBITAL ENERGIES

Orbital energies were calculated as follows. The ground-state (S₀) configurations of the molecules were computed using Density Functional Theory (DFT) with hybrid functional (B3LYP) and 6-31 g* basis set. For these closed shell systems (i.e., neutral molecules) the calculations were performed using the restricted approach, whereas for radical cations (open shell system containing an unpaired electron), the calculations were performed using the unrestricted approach. The energies of HOMO (highest occupied molecular orbital), SUMO (singly unoccupied molecular orbital for the radical cation) and LUMO (next unoccupied molecular orbital for the radical cation) were obtained from the ground-state geometries of the neutral molecule and the radical cation. Vibrational analysis on these geometries was performed and the lack of imaginary frequencies helped to ascertain the minima on the potential energy surface (PES). All calculations were performed using G09 suite of programs, as described in Frisch, M. J. T., G. W.; Schlegel, H. B.; Scuseria, G. E.; Robb, M. A.; Cheeseman, J. R.; Montgomery, Jr., J. A.; Vreven, T.; Kudin, K. N.; Burant, J. C.; Millam, J. M.; lyengar, S. S.; Tomasi, J.; Barone, V.; Mennucci, B.; Cossi, M.; Scalmani, G.; Rega, N.; Petersson, G. A.; Nakatsuji, H.; Hada, M.; Ehara, M.; Toyota, K.; Fukuda, R.; Hasegawa, J.; Ishida, M.; Nakajima, T.; Honda, Y.; Kitao, O.; Nakai, H.; Klene, M.; Li, X.; Knox, J. E.; Hratchian, H. P.; Cross, J. B.; Bakken, V.; Adamo, C.; Jaramillo, J.; Gomperts, R.; Stratmann, R. E.; Yazyev, O.; Austin, A. J.; Cammi, R.; Pomelli, C.; Ochterski, J. W.; Ayala, P. Y.; Morokuma, K.; Voth, G. A.; Salvador, P.; Dannenberg, J. J.; Zakrzewski, V. G.; Dapprich, S.; Daniels, A. D.; Strain, M. C.; Farkas, O.; Malick, D. K.; Rabuck, A. D.; Raghavachari, K.; Foresman, J. B.; Ortiz, J. V.; Cui, Q.; Baboul, A. G.; Clifford, S.; Cioslowski, J.; Stefanov, B. B.; Liu, G.; Liashenko, A.; Piskorz, P.; Komaromi, I.; Martin, R. L.; Fox, D. J.; Keith, T.; Al-Laham, M. A.; Peng, C. Y.; Nanayakkara, A.; Challacombe, M.; Gill, P. M. W.; Johnson, B.; Chen, W.; Wong, M. W.; Gonzalez, C.; and Pople, J. A; A.02 ed.; Gaussian Inc.: Wallingford Conn., 2009.

The orbital energies were as follows:

Orbital Energies for S101

| Molecule⁽¹⁾ | Form | Solvent | Orbital | Energy (eV) |
|---|---|---|---|---|
| S101 | neutral | anisole | HOMO | −4.8 |
| S101 | neutral | toluene | HOMO | −4.8 |
| S101 | neutral | anisole | LUMO | −1.0 |
| S101 | neutral | anisole | LUMO | −1.0 |
| S101 | neutral | anisole | triplet | 2.6 |
| S101 | neutral | toluene | triplet | 2.6 |
| S101 | radical cation | anisole | SUMO | −4.9 |
| S101 | radical cation | toluene | SUMO | −5.3 |
| S101 | radical cation | anisole | SUMO | −4.6 |
| | borate | | | |
| S101 | radical cation | toluene | SUMO | −4.7 |
| | borate | | | |
| S101 | radical cation | anisole | LUMO | −2.1 |
| S101 | radical cation | toluene | LUMO | −2.5 |
| S101 | radical cation | anisole | LUMO | −1.8 |
| | borate | | | |
| S101 | radical cation | toluene | LUMO | −1.9 |
| | borate | | | |

⁽¹⁾Orbital energies were computed for the core structure of S101 without the vinyl group.

Orbital Energies for S103

| Molecule⁽²⁾ | Form | Solvent | Orbital | Energy (eV) |
|---|---|---|---|---|
| S103 | neutral | anisole | HOMO | −4.9 |
| S103 | neutral | toluene | HOMO | −4.9 |
| S103 | neutral | Anisole | LUMO | −1.0 |
| S103 | neutral | Toluene | LUMO | −1.0 |
| S103 | neutral | Anisole | triplet | 2.6 |
| S103 | neutral | Toluene | triplet | 2.6 |
| S103 | radical cation | anisole | SUMO | −5.1 |
| S103 | radical cation | toluene | SUMO | −5.51 |
| S103 | radical cation | anisole | SUMO | −4.7 |
| | borate | | | |
| S103 | radical cation | toluene | SUMO | −4.9 |
| | borate | | | |
| S103 | radical cation | anisole | LUMO | −2.3 |
| S103 | radical cation | toluene | LUMO | −2.7 |
| S103 | radical cation | anisole | LUMO | −1.9 |
| | borate | | | |
| S103 | radical cation | toluene | LUMO | −2.1 |
| | borate | | | |

⁽²⁾Orbital energies were computed for the core structure of S103 without the vinyl group.

In both S101 and S103, the SUMO orbital energy of the radical cation is similar to the HOMO orbital energy of the neutral molecule. It is contemplated that this result means that when radical cations are mixed with neutral molecules, the radical cations will be able to act as a p-dopants, thus allowing the mixture to function as an HIL and/or as an HTL. The orbital energies shown in the table above can be used to design device architecture, including the use of specific materials for HIL, HTL, and EBL.

### EXAMPLE 11: TESTING OF OLED DEVICES

OLED devices were constructed as follows. Glass substrates (20 mm×15 mm) with pixelated tin-doped indium oxide (ITO) electrodes (Ossila Inc.) were used. The ITO was treated using oxygen plasma. For the HIL and/or HTL, each polymer was individually dissolved in electronic grade anisole (2% w/w) at elevated temperature (<100° C.) to ensure complete dissolution and passed through a 0.2 μm PTFE filter. The materials were deposited into a layer by dynamic spin coating whereby 20 μL of the solution was dispensed onto a spinning substrate. The spin speed (approximately 2000 RPM) was adjusted for each material to achieve a film thickness of approximately 40 nm. Some portions of the deposited film which covered sections of the electrodes were removed with toluene using a foam swab. The devices were then annealed at 205° C. for 10 minutes on a hot plate in an inert atmosphere. The emitting layer was a host/emitter mixture having 3 mole % emitter (Tris[3-[4-(1,1-dimethylethyl)-2-pyridinyl-κN][1,1′-biphenyl]-4-yl-κC]iridium) in a host (9-(4,6-Diphenyl-2-pyrimidinyl)-9′-phenyl-3,3′-bi-9H-carbazole).

The hole blocking layer (HBL), electron transport layer (ETL), and cathode were formed as follows. A 5 nm layer of 5-(4-([1,1′-biphenyl]-3-yl)-6-phenyl-1,3,5-triazin-2-yl)-7,7-diphenyl-5,7-dihydroindeno[2,1-b]carbazole as HBL material was deposited by thermal evaporation under high vacuum from an alumina crucible through an active area shadow mask. A 35 nm layer of 2,4-bis(9,9-dimethyl-9H-fluoren-2-yl)-6-(naphthalen-2-yl)-1,3,5-triazine as ETL material was deposited by thermal evaporation under high vacuum from an alumina crucible through an active area shadow mask. A 2 nm layer of lithium quinolate (liq) was deposited by thermal evaporation under high vacuum from an alumina crucible through a cathode shadow mask. A 100 nm layer of aluminum was deposited by thermal evaporation under high vacuum from a graphite crucible through a cathode shadow mask.

The OLED devices were tested as follows. Current-Voltage-Light (JVL) data was collected on unencapsulated devices inside a N₂ glovebox using a custom-made test board from Ossila Inc. The board contained two components: 1) X100 Xtralien™ precision testing source, and 2) Smart PV and OLED Board; in combination, these components were used to test OLED devices over a voltage range of −2 V to 7 V at increments of 0.1 V while measuring current and light output. The light output was measured using an eye response photodiode which includes an optical filter that mimics photopic eye sensitivity (Centronic E Series). The devices were placed inside of the testing chamber on the board and covered with the photodiode assembly. Electrical contact was made to the ITO electrodes by a series of spring-actuated gold probes inside of the Smart Board assembly. The photodiode was located at a distance of 3 mm above the ITO substrate. From the JVL data, critical device parameters were determined including the voltage required to reach 1000 cd/m² of brightness, the current efficiency (in cd/A) of the OLED at 1000 cd/m², and the driving voltage required to reach 10 mA/cm² of current in the OLED. A geometric factor was applied to the measured photodiode current to account for distance between the photodiode and the substrate (3 mm) and the relative positioning from each pixel on the substrate.

The accelerated service lifetime measurement involved the operation of an OLED under a constant current set to achieve an absolute brightness of 15000 cd/m² inside of a N₂ glovebox, without encapsulation. The devices were initially measured for their JVL performance. The device current was set to the required value to reach 15000 cd/m² of brightness and allowed to operate for a period of 15 minutes. The voltage to achieve the required driving current was allowed to vary throughout the test. The lifetime is the brightness after 15 minutes relative to the starting brightness.

Materials used were as follows:

p(S104)=a vinyl homopolymer of the following monomer:

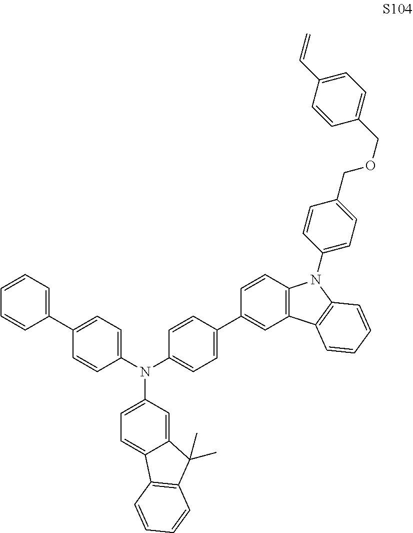 [SMILES: C=Cc1ccc(COCc2ccc(-n3c4ccccc4c4cc(-c5ccc(N(c6ccc(-c7ccccc7)cc6)c6ccc7c(c6)C(C)(C)c6ccccc6-7)cc5)ccc43)cc2)cc1]

p(S104) was not oxidized and contained no aminium radical.

The results of the testing were as follows.

| | | | | | | | life- |
|---|---|---|---|---|---|---|---|
| Exam- | | | | EFF | V1000 | V10 | time |
| ple | HIL | HTL | EBL | (%) | (V) | (V) | (%) |
| 13-1 | p(S101)-10 | p(S104) | none | 69.3 | 3.82 | 4.59 | 101.7 |
| 13-2 | p(S102)-10 | p(S104) | none | 71.3 | 3.83 | 4.67 | 100.3 |
| 13-3 | p(S103)-10 | p(S104) | none | 69.1 | 3.94 | 4.96 | 102.7 |

| | | | | | | |
|---|---|---|---|---|---|---|
| 13-4 | p(S101)-10 | none | 76.2 | 3.74 | 4.65 | 100.0 |
| 13-5 | p(S102)-10 | none | 74.2 | 3.81 | 4.61 | 104.3 |
| 13-6 | p(S103)-10 | none | 87.5 | 3.74 | 4.63 | 102.9 |
| 13-7 | p(S103)-10 | p(S103)-00 | 90.2 | 4.00 | 5.23 | 108.7 |

EFF = efficiency at brightness of 1000 candela/m² (higher values are desired)
V1000 = voltage at brightness of 1000 candela/m² (lower values are desired)
V10 = voltage at current of 10 mA (lower values are desired)
Lifetime = 100 × (lifetime of example)/(lifetime of Ex. 13-4)

Each of Examples 13-4, 13-5, and 13-6 had a single layer that served as both HIL and HTL.

All of the example diodes performed acceptably in all tests. Examples 13-4, 13-5, and 13-6 showed efficiency better than all the other examples. Examples 13-4, 13-5, and 13-6 showed V1000 lower than the other examples.

In additional tests (not shown), p(S101)-10, p(S102)-10, and p(S103)-10 were shown to perform acceptably when used as the HTL in an OLED.

1. An organic light-emitting diode comprising an anode layer, optionally one or more hole injection layers, one or more hole transport layers, optionally one or more electron blocking layers, an emitting layer, optionally one or more hole blocking layers, optionally one or more electron transport layers, an electron injection layer, and a cathode,
wherein either the hole injection layer, or the hole transport layer, or both of the hole injection layer and the hole transport layer, or a layer that functions as both a hole injection layer and a hole transport layer, comprises a polymer that comprises one or more triaryl aminium radical cations having the structure (S1) 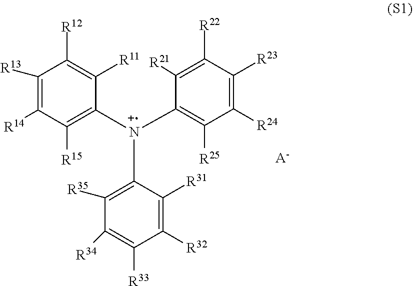
wherein each of R¹¹, R¹², R¹³, R¹⁴, R¹⁵, R²¹, R²², R²³, R²⁴, R²⁵, R³¹, R³², R³³, R³⁴, and R³⁵ is independently selected from the group consisting of hydrogen, deuterium, halogens, amine groups, hydroxyl groups, sulfonate groups, nitro groups, and organic groups, wherein two or more of R¹¹, R¹², R¹³, R¹⁴, R¹⁵, R²¹, R²², R²³, R²⁴, R²⁵, R³¹, R³², R³³, R³⁴, and R³⁵ are optionally connected to each other to form a ring structure;
wherein one or more of R¹¹, R¹², R¹³, R¹⁴, R¹⁵, R²¹, R²², R²³, R²⁴, R²⁵, R³¹, R³², R³³, R³⁴, and R³⁵ is covalently bound to the polymer, and
wherein A⁻ is an anion.

2. The diode of claim 1, wherein the diode comprises a dual-functional layer that functions as a hole injection layer and a hole transport layer, and wherein the diode does not comprise any additional hole injection layer or hole transport layer, and wherein the dual-functional layer comprises polymer that comprises one or more triaryl aminium radical cations having the structure (S1).

3. The diode of claim 2, wherein the diode additionally comprises one or more electron blocking layers.

4. The diode of claim 1, wherein the polymer is a vinyl polymer or a conjugated polymer.

5. The diode of claim 1, wherein the polymer additionally comprises one or more triaryl amine structures (S2) 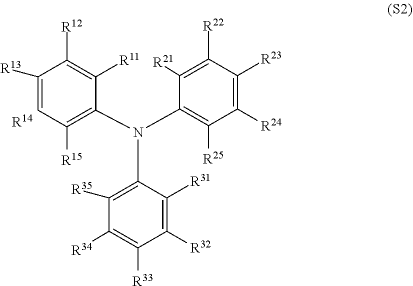
wherein each of R¹¹, R¹², R¹³, R¹⁴, R¹⁵, R²¹, R²², R²³, R²⁴, R²⁵, R³¹, R³², R³³, R³⁴, and R³⁵ is the same as in structure (S1).

6. The diode of claim 5, wherein the mole ratio of structures S2 to structures S1 is from 999:1 to 0.001:1.

7. The diode of claim 5, wherein the diode comprises a gradient layer that is located between the anode layer and the emitting layer and that comprises S1 groups and S2 groups, wherein the mole ratio of S2 groups to S1 groups is not uniform throughout the gradient layer.

8. The diode of claim 7, wherein the mole ratio of S2 groups to S1 groups in the portion of the gradient layer nearest the anode layer is defined as MRA:1, wherein the mole ratio of S2 groups to S1 groups in the portion of the gradient layer nearest the emitting layer is defined as MRE:1, and wherein MRA is less than MRE.

9. The diode of claim 8, wherein the ratio of MRA to MRE is 0.9:1 or less.

10. The diode of claim 1, wherein the composition additionally comprises one or more polymers that have no structure S1.

11. The diode of claim 1, wherein the polymer has number average molecular weight of 2,500 to 300,000 Da.

12. The diode of claim 1, wherein A⁻ is selected from the group consisting of BF₄⁻, PF₆⁻, SbF₆⁻, AsF₆⁻, ClO₄⁻, anions of structure SA, anions of structure MA, and mixtures thereof, wherein the structure SA is 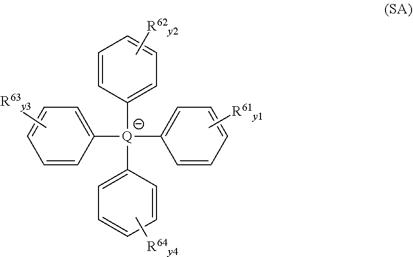
wherein Q is B, Al, or Ga, and wherein each of y1, y2, y3, and y4 is independently 0 to 5, and wherein each R⁶¹ group, each R⁶² group, each R⁶³ group, and each R⁶⁴¹ group is selected independently from the group consisting of deuterium, a halogen, an alkyl, and a halogen-substituted alkyl, and wherein any two groups selected from the R⁶¹ groups, the R⁶² groups, the R⁶³ groups, and the R⁶⁴¹ groups are optionally bonded together to form a ring structure, and wherein the structure MA is 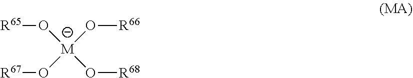
wherein M is B, Al, or Ga, and wherein each of R⁶², R⁶³, R⁶⁴, and R⁶⁵ is independently alkyl, aryl, fluoroaryl, or fluoroalkyl.
